SEQUENCE LISTING (26 sequences)

SEQ ID NO: 1 - DNA, 24 bp, synthetic construct
  source 1..24: mol_type = other DNA
gatcaagatcattgctcctcctga

SEQ ID NO: 2 - DNA, 21 bp, synthetic construct
  source 1..21: mol_type = other DNA
aagggtgtaaaacgcagctca

SEQ ID NO: 3 - DNA, 21 bp, synthetic construct
  source 1..21: mol_type = other DNA
tgatgaatcagggctgggatg

SEQ ID NO: 4 - DNA, 21 bp, synthetic construct
  source 1..21: mol_type = other DNA
attctgaagtctccgtgtccc

SEQ ID NO: 5 - DNA, 24 bp, synthetic construct
  source 1..24: mol_type = other DNA
gggatcagagatcggaagaagaaa

SEQ ID NO: 6 - DNA, 23 bp, synthetic construct
  source 1..23: mol_type = other DNA
aggaggtctgaacatcatcagtg

SEQ ID NO: 7 - DNA, 21 bp, synthetic construct
  source 1..21: mol_type = other DNA
cagccaagtctgtgacttgca

SEQ ID NO: 8 - DNA, 21 bp, synthetic construct
  source 1..21: mol_type = other DNA
gtgtggaatcaacccacagct

SEQ ID NO: 9 - DNA, 26 bp, synthetic construct
  source 1..26: mol_type = other DNA
actctgtctccttcctcttcctacag

SEQ ID NO: 10 - DNA, 22 bp, synthetic construct
  source 1..22: mol_type = other DNA
cactgcccaacaacaccagctc

SEQ ID NO: 11 - DNA, 22 bp, synthetic construct
  source 1..22: mol_type = other DNA
agcctgggcatccttgagttcc

SEQ ID NO: 12 - DNA, 26 bp, synthetic construct
  source 1..26: mol_type = other DNA
tcatagaaccattttcatgctctctt

SEQ ID NO: 13 - DNA, 26 bp, synthetic construct
  source 1..26: mol_type = other DNA
tcaacttacgacgagtttatcaggaa

SEQ ID NO: 14 - DNA, 23 bp, synthetic construct
  source 1..23: mol_type = other DNA
gaagagaatctccgcaagaaagg

SEQ ID NO: 15 - DNA, 23 bp, synthetic construct
  source 1..23: mol_type = other DNA
tccatccagtggtttcttctttg

SEQ ID NO: 16 - RNA, 20 nt, synthetic construct
  source 1..20: mol_type = other RNA
gcttcatgtggtcggggtag

SEQ ID NO: 17 - RNA, 20 nt, synthetic construct
  source 1..20: mol_type = other RNA
cacgagggtgggccagggca

SEQ ID NO: 18 - RNA, 20 nt, synthetic construct
  source 1..20: mol_type = other RNA
agggtcttctctactcaagg

SEQ ID NO: 19 - RNA, 20 nt, synthetic construct
  source 1..20: mol_type = other RNA
ttattactagagtagcaggg

SEQ ID NO: 20 - RNA, 20 nt, synthetic construct
  source 1..20: mol_type = other RNA
ggagcagatggtcactccag

SEQ ID NO: 21 - RNA, 20 nt, synthetic construct
  source 1..20: mol_type = other RNA
gtcattgtgtctgttcgggg

SEQ ID NO: 22 - RNA, 20 nt, synthetic construct
  source 1..20: mol_type = other RNA
tactgccgtgtatcgtattg

SEQ ID NO: 23 - RNA, 20 nt, synthetic construct
  source 1..20: mol_type = other RNA
ttgtataggaccctcgggca

SEQ ID NO: 24 - RNA, 20 nt, synthetic construct
  source 1..20: mol_type = other RNA
catctcaccagcctagcagg

SEQ ID NO: 25 - RNA, 20 nt, synthetic construct
  source 1..20: mol_type = other RNA
tcattaacccaggagccacg

SEQ ID NO: 26 - RNA, 20 nt, synthetic construct
  source 1..20: mol_type = other RNA
acctgcttcacagataactg

(19) United States
(12) Patent Application Publication
(10) Pub. No.: US 2024/0358008 A1
(43) Pub. Date: Oct. 31, 2024

(54) COMPOSITIONS AND METHODS FOR SWITCHING ANTIBIOTIC RESISTANCE MARKERS PROGRESSIVELY FOR INTEGRATION (mSwAP-In)

(71) Applicant: New York University, New York, NY (US)

(72) Inventors: Weimin ZHANG, New York, NY (US); Jef BOEKE, New York, NY (US)

(21) Appl. No.: 18/686,704

(22) PCT Filed: Aug. 31, 2022
(86) PCT No.: PCT/US2022/075749

§ 371 Date: Feb. 26, 2024

Related U.S. Application Data
(60) Provisional application No. 63/239,339, filed on Aug. 31, 2021.

(51) Int. Cl. A01K 67/0278; A61K 48/00; C12N 5/0735; C12N 9/22; C12N 9/48; C12N 15/11; C12N 15/90

(52) U.S. Cl. CPC A01K 67/0278; A61K 48/0066; C12N 5/0606; C12N 9/22; C12N 9/485; C12N 15/11; C12N 15/907; C12Y 304/17023; A01K 2217/072; A01K 2227/105; C12N 2310/20

(57) ABSTRACT

Provided are compositions, methods, and systems referred to as “mSwAP-In” which stands for mammalian Switching Antibiotic resistance markers Progressively for Integration. The compositions, methods, and systems are capable of iteratively overwriting one or a series or genes with a synthetic DNA cargo.

### CROSS REFERENCE TO RELATED APPLICATIONS

This application claims priority to U.S. provisional patent application No. 63/239,339, filed Aug. 31, 2021, the entire disclosure of which is incorporated herein by reference.

### STATEMENT REGARDING FEDERALLY SPONSORED RESEARCH

This invention was made with government support under grant number RM1-HG009491 awarded by the National Institutes of Health. The government has certain rights in the invention.

### FIELD

The present disclosure relates generally to DNA editing, and more particularly to compositions and methods used for inserting large pieces of DNA into a variety of eukaryotic cell types.

### SEQUENCE LISTING

The instant application contains a Sequence Listing which has been submitted in xml format and is hereby incorporated by reference in its entirety. Said.xml copy was created on Aug. 31, 2022, is named “058636_00554_ST26.xml”, and is 23,851 bytes in size.

### BACKGROUND

Developing a genome writing method can help researchers to introduce a large number of design features into a mammalian genome in a single delivery step, which is extremely challenging for “one-edit-at-a-time” methods. Existing big DNA delivery methods such as Inducible Cassette Exchange (ICE), Recombinase-mediated genomic replacement (RMGR), and other related methods have drawbacks. There is a need for a mammalian genome writing method that is scarless, iterable and functionally homozygous. The present disclosure is pertinent to this need.

### BRIEF SUMMARY

The present disclosure provides compositions, methods and systems referred to herein as “mSwAP-In” which stands for mammalian Switching Antibiotic resistance markers Progressively for Integration. Non-limiting embodiments of the mSwAp-In approach are illustrated in the Figures. For example a general overview of the mSwAP-In compositions and methods is provided in the panels of FIG. 1, with additional compositions and method steps illustrated in the panels of FIGS. 2, 3 and 4. The described approach allows sequential modification of chromosomes in eukaryotic cells by iterative insertion of marker cassettes, referred to as MC1 and MC2. The cassettes include one or more DNA cargoes, and are manipulated using any of a variety of nucleases to promote iterative and mutually exclusive homologous recombination events, which are in part facilitated homology arms. The DNA cargoes can include upstream regulatory elements, downstream regulatory elements, intronic elements, and combinations thereof. Other components of the described compositions and method include but are not limited to the use of custom site cuts, as well as positive and negative selection to facilitate selection of cells that contain integrated cargoes in an iterative manner. The mSwAP-In approach also provides universal gRNA target that are orthogonal to all mammalian sequences. The approach is configured such that inserted cargo DNA sequences do not include the UGT sequence from prior steps, thereby avoiding cleavage of previously introduced DNA cargo.

The compositions and methods permit overwriting any existing DNA sequence of any length selected by a user of the described system. The method can be performed in a scarless manner, meaning that, other than introduced DNA cargo, no remnants of the mSwAP-In compositions remain in the chromosome(s) after the method is performed. The disclosure includes repeating the described methods for any number of times. Thus, serial modifications are provided. In embodiments, the serial modifications can result in serial humanization of non-human chromosomes. The method is applicable to any single chromosome, and to chromosome pairs. Thus, mSwAP-In can be used to produce homozygous, heterozygous, or hemizygous modifications. The described compositions and methods can be used to modify any type of eukaryotic cells, including but not necessarily limited to mammalian cells. Likewise, the cells that are modified include but are not limited to totipotent, pluripotent, multipotent, or oligopotent stem cells when the modification is made. By using the described compositions and methods, modified non-human mammals can be made that contain chromosomes that contain integrated heterologous DNA cargoes. Such integrated DNA cargoes may be from or derived from a human genome, or any other source of DNA. Thus, the disclosure provides for making non-human mammals that can be used, for example, to study human genes in an in vivo context, produce products encoded by human genes using non-human modified mammals or other cell types, and a variety of other purposes that will be evident to those skilled in the art given the benefit of this disclosure.

### BRIEF DESCRIPTION OF THE FIGURES

FIG. 1. mSwAP-In workflow overview. (Panel A). Two interchangeable marker cassettes underlie mSwAP-In selection and counter selection (Panel B). Stepwise genome rewriting using mSwAP-In. A prior engineering step to delete endogenous Hprt enables later iteration. Step 1: integrate marker cassette 1 upstream of locus of interest. Step 2: deliver payload DNA and Cas9-gRNAs for integration through homologous recombination. Step 3: deliver next payload DNA following same strategy as Step 2, swapping back to marker cassette 1. Iterative Steps 2 and 3 can be repeated indefinitely using a series of synthetic payloads, by alternating selection for marker cassettes (curved arrows). Step 4: remove final marker cassette. YAC: yeast artificial chromosome, BAC: bacterial artificial chromosome, 6-TG: 6-Thioguanine, GCV: ganciclovir. Gray bars are native chromosome regions, purple bars are synthetic incoming DNAs. Blue and brown scissors are universal Cas9-gRNAs cutting UGT1 and UGT2, respectively; gray scissors are genomic-targeting Cas9-gRNAs. Superscript R, drug resistance.

FIG. 2. Reversibly knock out Piga gene. (Panel A) Piga transcript is blocked by a floxed stop cassette, which can be reversed by transient expression of Cre recombinase to excise the stop cassette. (Panel B) Wild-type mESC with functional Piga gene is sensitive to proaerolysin, Piga reversibly knock out clones were resistant to proaerolysin.

FIG. 3. Three mSwAP-In genome writing scenarios. (Panel A) When MC1 is inserted into both wild-type alleles in the first step, both wild-type alleles can be replaced by their synthetic counterparts, thus producing a homozygously edited genome. Or one allele is replaced by its synthetic counterpart, the other allele is deleted by non-homologous end joining DNA repair mechanism, thus producing hemizygous genome. P1 and P2 are two primers flank the cutting sites of endonuclease(s), which can be used to distinguish biallelic and hemizygous genome writing clones. (Panel B) When MC1 is inserted into one of the two wild-type alleles, only that allele will be replaced by the synthetic DNA, thus produce a heterozygously edited genome.

FIG. 4. Representative embodiment for obtaining biallelically edited clones when the gene of interest is autosomal.

FIG. 5. Schematic diagrams show the ACE2 humanization design. Two constructs for ACE2 were designed with components including UGT1, mHA, hACE2, MC2, mHA, UGT1. UGT1, universal gRNA target 1. mHA, mouse homology arm. MC, marker cassette. DNase signal and H3K27 acetylation tracks are from UCSC genome browser.

FIG. 6. Marker cassette 1 (top schematic) insertion verification by junction PCR (bottom gel photograph).

FIG. 7. Genotyping of mESC clones after the delivery of 116 kb hACE2 payload (Panel A) and 180 kb hACE2 payload (Panel B). (Panel C) hACE2 copy number determination by qPCR. The ratio between hACE2 and mActb is 0.5, indicating a single copy of human ACE2 was delivered to the X chromosome of a male mESC line, as expected. Copy number was normalized to the mActb gene. (Panel D) Sequencing coverage of 116 kb hACE2 and 180 kb hACE2 mSwAP-In clones. Reads were mapped to hg38 (up) and mm10 (bottom).

FIG. 8. Genotyping of 116 kb ACE2 humanized mouse produced by tetraploid complementation. Various tissues were collected for genomic DNA extraction. 14 primer sets were used for each sample. Primer locations were indicated in the upper panel schematic.

FIG. 9. hACE2, mAce2 expression levels in liver, spleen, lung, kidney, small intestine, heart, brain, large intestine and testis were detected by RT-qPCR in hACE2 mice (left) and wild-type mice (right). Expression was normalized to mouse Actb. Bars represent the mean±SD of three technical replicates.

FIG. 10. hACE2 transcript variants detection. (Panel A) RT-PCR detection of dACE2 isoform (transcript variant 5) in various tissues of hACE2 mouse. cACE2, canonical A (E2 transcript. (Panel B) RT-PCR detection of hACE2 transcript 3 in various tissues of hACE2 mouse.

FIG. 11. Schematic diagram showing the design of TP53-WRAP53 humanization. The dashed line box in the mouse genome panel indicates the mouse genome segment to be deleted, the dashed line box in the human genome panel indicates the human genome segment that to be delivered. H3K27Ac track is from UCSC genome browser. MC, marker cassette.

FIG. 12. Marker cassette 1 biallelic insertion (top schematic) verification by junction PCR (bottom gel photographs).

FIG. 13. Humanizing TP53-WRAP53 via biallelic mSwAP-In. (Panel A) Genotyping PCR of TP53-WRAP53 humanized clones. Panel (Panel B) Capture sequencing of two candidates and a wild-type control, sequencing reads were aligned to human reference genome (top) and mouse reference genome (bottom).

FIG. 14. Detection of hemizygosity that mediated by biallelic mSwAP-In. (Panel A) Large segment deletion can be detected using a pair of primer (P1-P2) that flank the endonuclease cutting sites. Clones marked by triangle symbols are hemizygous. (Panel B) Upper panel, the basis of the plasmid standard for qPCR assay. Bottom panel, quantitative PCR of plasmid standard and the genomic DNA of the engineered mESCs. (Panel C) Representative mouse tail biopsy genotyping PCR using the 8 pairs of primers annotated in FIG. 13 (small arrowheads), panel A. m: mouse PCR amplicons, h: human amplicons

FIG. 15. hTP53 expression level in TP53-WRAP53 humanized mice. (Panel A) A reference plasmid containing the qPCR region of TP53, Trp53 and Actb genes. (Panel B) hTP53 and mTrp53 expression levels in 26 tissues of human (left) or mouse (right). (Panel C) hTP53 and mTrp53 expression levels in wild-type, homozygous and heterozygous TP53-WRAP53 tissues. pWZ783 is the reference plasmid shown in panel A.

FIG. 16. Human p53 isoforms detection in TP53-WRAP53 humanized mice. Primer used for the RT-PCR are listed in Table 2.

FIG. 17. Serial humanization of TMPRSS2 in ACE2 humanized mESCs. (Panel A) Schematic of TMPRSS2 humanization design. Top, mouse Tmprss2 gene locus, grey box highlights the region to be replaced by human TMPRSS2 gene. Bottom, human TMPRSS2 gene locus, grey box highlights the genomic region used for the humanization. (Panel B) Two of MC1 biallelic insertion scenarios. Left, MC1 is inserted into both alleles. Right, MC1 is inserted in only one allele, the other allele has a deletion caused by non-homologous end joining, which eliminate the binding site of either primer 1 or primer 4. (Panel C) A reference plasmid has one copy of marker cassette 1 and one copy of Actb gene. (Panel D) MC1 copy number qPCR. Six MC1 insertion clones that have both left and right junctions and lack the amplicon from primer 1-4 were used for MC1 copy number qPCR. Primer 5 and 6 were used for MC1 qPCR, primer 7 and 8 were used for Actb qPCR. (Panel E) Full scheme for serial humanization shows that MC2 is left at ACE2. MC1 is inserted in place of mTmprss2. MC2. 1 is used as the selection marker for mSwAP-In of hTMPRSS2 gene and differs from MC2 in that it contains the neo gene rather than the bsd gene as positive selection marker.

FIG. 18. Biallelic humanization of TMPRSS2. (Panel A) Success rates of biallelic humanization in three MC1 founder lines. (Panel B) TMPRSS2 copy number determination by qPCR. A reference plasmid containing one copy of human TMPRSS2 gene and one copy of mouse Actb gene was used. (Panel C) Sequencing coverage of TMPRSS2 humanized mES clones. Reads were mapped to hg38 (up) and mm10 (bottom).

FIG. 19. ACE2. TMPRSS2 mice production and TMPRSS2 expression. (Panel A) ACE2 TMPRSS2 both genes humanized mouse model establishment by tetraploid complementation. Representative genotyping PCR showing the absence of mAce2 and mTmprss2, presence of hACE2 and hTMPRSS2. (Panel B) Human TMPRSS2 expression pattern in ACE2+TMPRSS2 humanized mouse (left), mouse Tmprss2 expression pattern in ACE2 only humanized mouse (right).

FIG. 20. Wild-type Trp53 overwritten by synthetic “CG less” Trp53. (Panel A) Design of the synthetic “CG less” Trp53; five p53 mutation hotspots were recoded to AGA to eliminate the CG dinucleotides. Heterozygous mSwAP-In (Panel B) and homozygous or hemizygous mSwAP-In (Panel C) were demonstrated. Efficiencies were indicated in the chart on right-hand side; n is the number of clones that passed genotypic screening, and were verified by Sanger sequencing. (Panel D) Quantitative PCR assay to determine the copy number of “CG less” Trp53, two primer sets were used for each clone. (Panel E) Capture sequencing of biallelic mSwAP-In engineered mES clones, the black double headed arrows indicate the regions of large fragment deletion. Dashed line box region is the engineered genomic segment.

FIG. 21. Second round of mSwAP-In at mouse Trp53 locus. (Panel A) Sequencing reads from synTrp53 and three payload constructs are aligned to mm 10 reference. Genes are shown by horizontal arrows. Vertical arrows indicate the position of the 8 mPCRTags interspersed in the payloads. (Panel B) The design of synthetic and wild type specific primers. The synthetic specific forward primer sits on the mPCRTag while the wild type specific forward primer skips the mPCRTag leaving 4-6 bp in the 3′ end for annealing. (Panel C) Pulsed field gel electrophoresis of three linearized payload constructs. (Panel D) mPCRTagging result shows the heterozygous and homozygous/hemizygous mSwAP-In pattern for three payloads. (Panel E) Summary of mSwAP-In success rate. n=46 in each group.

FIG. 22. Marker cassette removal. (Panel A) Schematic illustration of MC1 removal in heterozygous and homozygous formats. (Panel B) Positions of genotyping primers, successful MC1 removal clones show absence of P1-P2, P3-P4, presence of P1-P4. (Panel C) Summary of marker cassette 1 removal efficiency.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value is optional and means for example +/−10%.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included.

The disclosure includes all polynucleotide and all amino acid sequences that are identified herein by way of a database entry. Such sequences are incorporated herein as they exist in the database on the effective filing date of this application or patent.

Representative compositions and methods are provided. The disclosure includes all compositions and steps as described herein and as shown in the accompanying figures. The disclosure includes the proviso that any single or combination of reagents and steps may be excluded. Some or all the steps may be performed sequentially or concurrently. The disclosure includes all compositions of matter formed during performance of the described method. The disclosure includes all expression vectors and combinations of expression vectors used in the described methods and systems, all guide RNAs, and all engineered guide RNA recognition sequences that are used, for example, during performance of the described methods.

In embodiments, the described compositions, methods and systems of the disclosure are referred to herein from time to time as “mSwAP-In” which stands for mammalian Switching Antibiotic resistance markers Progressively for Integration. While the described acronym contemplates use in mammalian cells, it is considered that mSwAP-In may be used for modifying one or more chromosomes of any eukaryotic cells, including but not necessarily limited to fungi such as yeasts, other eukaryotic microorganisms including but not limited to eukaryotic parasites, plant cells, insect cells, and cells of any other non-mammalian animals, including but not necessarily limited to cells of avian animals, fish and worms.

In embodiments, the cells that are modified by the approaches of this disclosure are totipotent, pluripotent, multipotent, or oligopotent stem cells when the modification is made. The stem cells may exhibit the described potency naturally, or the stem cells may be induced stem cells. In embodiments, the cells are hematopoietic stem cells. In embodiments, the cells are embryonic stem cells, or adult stem cells. In embodiments, the cells are epidermal stem cells or epithelial stem cells or neural stem cells. In embodiments, the cells are cancer cells, or cancer stem cells. In embodiments, the cells are spermatogonial stem cells. In embodiments, the cells are muscle cells, skin cells, retinal cells, or precursors of any tissue or organ. In embodiments, the cells are differentiated cells when the modification is made. In embodiments, the cells are leukocytes. In embodiments, the leukocytes are of a myeloid or lymphoid lineage.

In embodiments, the disclosure includes obtaining cells from an individual, modifying the cells ex vivo using a system as described herein, and reintroducing the cells or their progeny into the individual or an immunologically matched individual for prophylaxis and/or therapy of a condition, disease or disorder. In embodiments, the cells modified ex vivo as described herein are autologous cells. In embodiments, the cells are provided as cell lines. In embodiments, the cells are engineered to produce a protein or other compound, and the cells themselves and/or the protein or compound they produce is used for prophylactic or therapeutic applications.

In embodiments, cells modified using mSwAP-In are mammalian cells and as such may be from any mammal, non-limiting embodiments of which include humans, any member of the order Rodentia including but not limited to mice and rats, ferrets, as well as canine animals, feline animals, equine animals, bovine animals, and porcine animals. In embodiments, the disclosure provides methods for making modified non-human mammals, including but not necessarily limited to modified mice. Modified non-human animals are included within the scope of the disclosure. In embodiments, modified non-human animals comprise one or more intact human genes or functional segments thereof inserted into one or more chromosomes using the mSwAP-In system. In embodiments, the disclosure relates to producing modified mice that comprise one or more human or non-human genes. In embodiments, the modified mice comprise a replacement of all or a segment of a mouse gene with a human gene.

In embodiments, the disclosure comprises providing a treatment to an individual in need thereof by introducing a therapeutically effective amount of modified eukaryotic cells as described herein to the individual, such that the payload produces a polynucleotide, peptide, protein, a drug, a prodrug, an immunological agent, an enzyme, or any other agent that may have a beneficial effect. A corrected or new gene may also be considered a therapeutic agent.

In embodiments, the modified eukaryotic cells can be provided in a pharmaceutical formulation, and such formulations are included in the disclosure. A pharmaceutical formulation can be prepared by mixing the modified eukaryotic cells with any suitable pharmaceutical additive, buffer, and the like. Examples of pharmaceutically acceptable carriers, excipients and stabilizers can be found, for example, in Remington: The Science and Practice of Pharmacy (2012) 22nd Edition, Philadelphia, PA. Lippincott Williams & Wilkins, the disclosure of which is incorporated herein by reference.

As further described herein, mSwAP-In provides for sequential modification of eukaryotic cells by iterative insertion of marker cassettes (MC1 and MC2) and DNA cargoes. The compositions and methods permit overwriting any existing DNA sequence that comprises any sequence and length selected by a user of the described system.

In more detail, mSwAP-In comprises an iterative method that can be repeated indefinitely to thereby introduce large DNA molecules, including but not necessarily limited to a library of different DNA molecules, into one or more eukaryotic chromosomes, within the same locus, or at different loci. Further, the method can be performed in a scarless manner, meaning that, other than introduced DNA cargo, no remnants of the mSwAP-In compositions remain in the chromosome(s) after the method is performed. Additionally, mSwAP-In can be used to produce homozygous, heterozygous, or hemizygous modifications. In an embodiment, the described approach provides for functionally homozygous genome writing, meaning that only functional copy(s) of the delivered DNA exist in the modified cells and the pre-existing native form of the DNA is eliminated as part of the delivery process.

Certain aspects of the disclosure are illustrated in the figures. The figures illustrate, among other elements, configurations of input DNA constructs, nuclease cleavage sites, and locations of homologous recombination. The figures depict representative constructs which have various features shown in a left to right orientation. The features shown to the left and right of other features thus depict upstream (e.g., to the left) and downstream (e.g. to the right) segments, relative to one another. The disclosure includes alternative constructs with different orientations. For instance, the Marker Cassette 1 (MC1), described further below, may be positioned such that it is upstream (to the left) of a target gene, or downstream (to the right) of a target gene. Those skilled in the art will recognize, given the benefit of the present disclosure that, depending on the relative location of, for example, MC1, the remaining inputs and steps will have positions that are coordinated in an iterative process relative to one another so that MC1 and MC2 are swapped in a stepwise fashion. The designation of “MC1” and “MC2” is therefore arbitrary, and does not necessitate a sequential MC1, MC2 order of constructs or steps, provided MC1 and MC2 are different from one another.

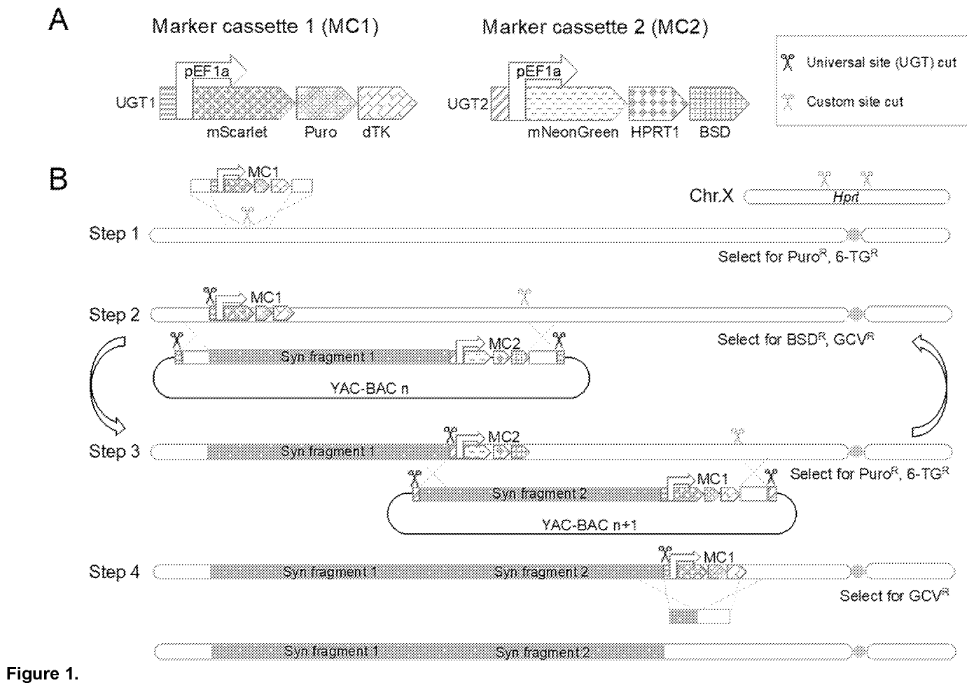

In particular, the two marker cassettes (MC1 and MC2) discussed above as used in performing mSwAP-In each comprise the following components: 1) a promoter; 2) a sequence encoding any detectable marker, such as a fluorescent marker (illustrated in a non-limiting embodiment by mScarlet-I gene in MC1 and mNeonGreen gene in MC2, thereby demonstrating that the detectable marker in MC1 is different from the detectable marker in MC2), 3) a positive selection marker (illustrated in a non-limiting embodiment by puromycin resistance gene in MC1 and blasticidin resistance gene in MC2, thereby demonstrating that the positive selection marker in MC1 is different from the positive selection marker in MC2, and 4) a negative selection marker (illustrated by delta thymidine kinase [dTK] gene in MC1 and the coding sequence of the human HPRT1 gene in MC2, thereby demonstrating that the negative selection marker in MC1 is different from the negative selection marker in MC1. See, for example, FIG. 1, panel A.

In the described marker cassettes any suitable promoter that is operably linked to a sequence that is transcribed can be used. A representative promoter used in the Examples comprises the EF1α promoter. Alternatives include but are not limited to a CAG promoter, a phosphoglycerate kinase (PGK) gene promoter, a tetracycline response element promoter (TRE), a simian virus 40 (SV40) promoter, a cytomegalovirus (CMV) promoter, and a polyubiquitin C (Ubc) promoter. The sequences of all of these promoters are known in the art and can be included in the described marker cassettes using ordinary skill when given the benefit of the present disclosure. Likewise, any suitable transcription termination signal can be included at the end of any sequence that is transcribed. A variety of transcription termination signals are known in the art.

To promote homologous recombination efficiency in mammalian cells, the disclosure provides two universal gRNA target (UGT) sequences at the left end of each marker cassette that are orthogonal to all mammalian sequences. By “orthogonal” it is mean that the UGT sequences do not appear in the chromosome prior to being modified using mSwAP-In. Further, the inserted cargo DNA sequences do not include the UGT sequence from prior steps to avoid cleaving the previously introduced DNA cargo. Using this design, the UGTs can be used in all mSwAP-In steps at any target locus. Representative UGT sequences are identified below by way of guide RNA sequences which target the UGT sequences. The disclosure provides for use of any suitable UGT and companion guide RNA sequences, provided the UGT and companion guide RNA sequences do not target the endogenous genome.

Representative and non-limiting embodiments of constructs and steps of this disclosure are generally illustrated in FIGS. 1 panels A and B. The UGT is as described above and in the Examples. The UGT is recognized by a guide RNA-directed nuclease or a non guide RNA-directed nuclease. Working examples are shown using Cas9, but other guide RNA-directed Cas enzymes can also be used. In embodiments, the Cas enzyme is any of Type I, Type II, Type III, Type IV, Type V or Type VI Cas enzyme. In a non-limiting embodiment, the Cas enzyme may be any Cas9, a non-limiting example of which comprises Streptococcus pyogenes Cas9 (SpCas9). Derivatives of Cas9 are known in the art and may also be used. Such derivatives may be, for example, smaller enzymes than Cas9, and/or have different proto adjacent motif (PAM) requirements. In a non-limiting embodiments, the Cas enzyme may be Cas12a, also known as Cpf1, or SpCas9-HF1, or HypaCas9. Other nucleases can be used instead of Cas nuclease, examples of which include TALENS, zinc-finger nucleases and MADzymes. Non-limiting examples of MADzymes known in the art include MAD2 and MAD7 and are included in the Cas12a category of nucleases.

FIG. 1 panels A and B also show use of “custom site cuts” illustrated by gray scissors which target the sequence of interest in the genome which is to be replaced. These cuts may be made using any of the Cas enzymes described above, but do not necessarily need to be made by a guide-RNA directed Cas enzyme. The custom cut sites are from endogenous genome but orthogonal to the payload DNA.

The figures and examples of the disclosure describe use of positive and negative selection. Positive selection may be used alone, or concurrently with negative selection. Likewise, negative selection may be used alone, or concurrently with positive selection. Various specific selectable markers are demonstrated in the examples, but other selectable markers are known in the art and can be adapted for use in embodiments of the disclosure. In embodiments, positive selection markers include but are not limited to puromycin N-acetyltransferase (pac), Blasticidin S deaminase (bsd), Neomycin (G418) resistance gene (neo), Hygromycin resistance gene (hygB), Zeocin resistance gene (Sh ble) and hypoxanthine phosphoribosyltransferase 1 gene (HPRT1). In embodiments, non-limiting examples of negative selection markers include the herpes simplex virus type 1-thymidine kinase (HSV1-TK) gene that renders cells sensitive to ganciclovir (GCV) by converting it to the toxic metabolite GCV-triphosphate (GCV-TP). The human HPRT1 gene that confers 6-thioguanine sensitivity by converting 6-thioguanine to 6-thioguanosine monophosphate (TGMP). The cytosine deaminase gene (codA) converts 5-fluorocytosine (5-FC) into toxic metabolite 5-fluorouracil (5-FU), which can be used as negative selection marker.

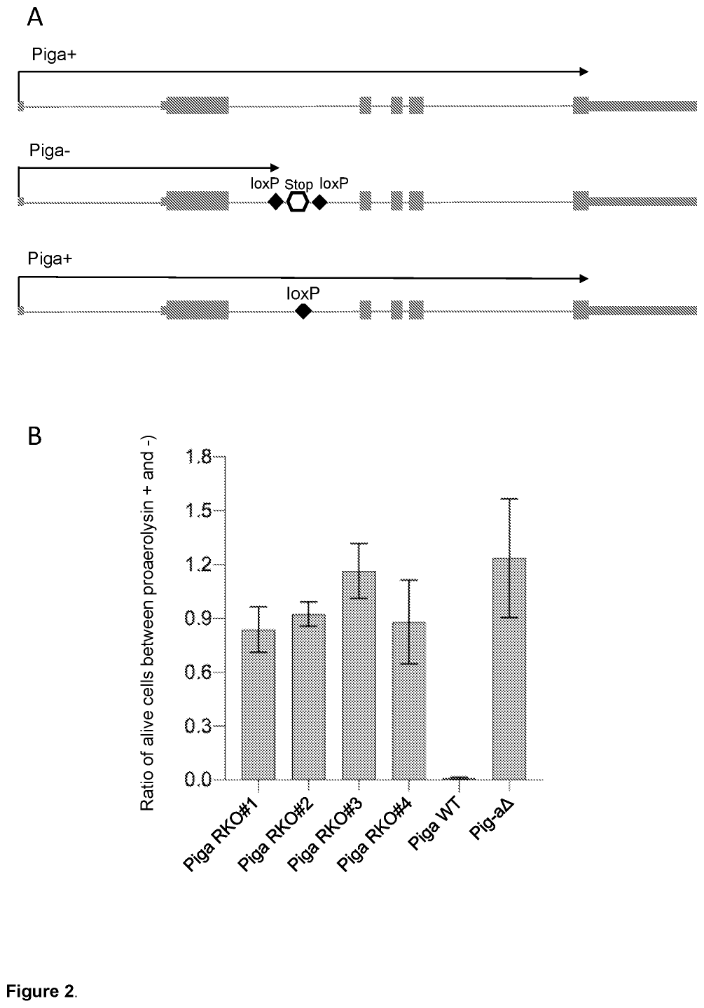

In another embodiment, a PIGA-based selection can be used. In general, cells subjected to mSwAP-In may include, at least transiently, a mutated X-linked PIGA (phosphatidylinositol glycan class A) gene. A mutation in the PIGA gene, and its repair, may be made using any suitable techniques, including but not necessarily limited to CRISPR and recombinase-mediated approaches that include homologous recombination. The protein encoded by the wild type PIGA gene renders cells sensitive to the bacterial prototoxin proaerolysin, also enables the binding of glycosylphosphatidylinositol (GPI)-anchored proteins to the cell membrane, making the cells distinguishable from PIGA null or PIGA mutation cells. Thus, cells that are subjected to mSwAP-In may be selected to both negative and positive selection depending on the mutation status and/or presence of the PIGA gene in the cells. In embodiments, a PIGA gene modification comprises a reversible PIGA gene knockout (FIG. 2). A similar approach can be adapted with the endogenous mouse (or other mammalian cells) using the Hprt gene, the protein product of which confers resistance to 6-thioguanine. In a related embodiment, a mutation of a PIGL gene can be used. A representative approach to use of PIGA and PIGL as selection markers is described in Li D, et al., Application of counter-selectable marker PIGA in engineering designer deletion cell lines and characterization of CRISPR deletion efficiency. Nucleic Acids Res. 2021 Mar. 18; 49 (5): 2642-2654, from which the disclosure is incorporated herein by reference.

The figures and examples of the disclosure describe use of detectable markers. Any detectable markers can be used, non-limiting examples of which include green fluorescent protein (GFP), enhanced GFP, mCherry, mTAGBFP2, mPlum, YFP, mPapaya, mStrawberry, blue fluorescent protein (BFP), Halo tag, Sirius, and the like. In embodiments, the detectable marker produces a signal that comprises UV light (<380 nm), visible light (380-740 nm) or far red (>740 nm).

As described further below and by way of the figures, the described MC1 and MC2 constructs include homology arms. The sequence of the 5′ and 3′ homology arms are not particularly limited, provided they have a length that is adequate for homologous recombination to occur when nuclease-mediated cleavage of the selected locus occurs. In embodiments, the 5′ and 3′ homology arms have a length of from 100 base pairs (bp)-2 kilobases (kb), inclusive, and including all integers and ranges of integers there between.

The DNA cargo introduced into chromosomes using mSwAP-In can comprise any DNA sequence. In embodiments, the DNA cargo sequence is heterologous to the cells that are modified by mSwAP-In. “Heterologous” means the cells did not contain the cargo DNA sequence prior to being modified by mSwAP-In. The Examples below demonstrate introduction of cargo DNA that is up to 180 kb, but it is expected that much larger sequences can be introduced using the iterative mSwAP-In approach. Yeast cells can be used to host mammalian DNAs in the form of YACs (yeast artificial chromosomes) of at least 1 megabase (Mb) and thus the disclosure comprises introducing fragments up to this length, or more, in a single step using mSwAP-In.

In embodiments, the DNA payload is heterologous but is from the same species as the mSwAP-In modified cells. For example, mSwAP-In can be used to correct or otherwise change a gene, but not change the species origin of the gene. In embodiments, the DNA payload is from a different species as compared to the mSwAP-In modified cells. In embodiments, the disclosure provides for insertion of large DNA molecules in one or more selected loci, wherein the large DNA molecules may comprise regulatory signals, including but not necessarily limited to promoters, enhancers, and the like. Due to the capability of mSwAP-In to introduce large DNA molecules, the regulatory elements may be distant relative to a gene, transcription unit, etc., that may also be introduced as part of the DNA payload.

The DNA cargoes may be devoid of any sequence that can be transcribed, and as such may be transcriptionally inert. Such sequences may be used, for example, to alter a regulatory sequence in a genome, e.g., a promoter, enhancer, miRNA binding site, or transcription factor binding site, to result in knockout of an endogenous gene, or to provide an interval in the chromosome between two loci, and may be used for a variety of purposes, which include but are not limited to treatment of a genetic disease, enhancement of a desired phenotype, study of gene effects, chromatin modeling, enhancer analysis, DNA binding protein analysis, methylation studies, and the like.

In embodiments, the DNA payload comprises a sequence that may be transcribed by any RNA polymerase, e.g., a eukaryotic RNA polymerase, e.g., RNA polymerase I, RNA polymerase II, or RNA polymerase III. In embodiments, the RNA that is transcribed may or may not encode a protein, or may comprise a segment that encodes a protein and a non-coding sequence that is functional, such as a functional mRNA. In embodiments, the DNA payload comprises one or more splice junctions.

In embodiments, and as further discussed herein, the DNA payload comprises an intact gene, or a gene fragment. In embodiments, the DNA payload comprises more than one gene.

One or more of the described constructs can be provided in any suitable form. In embodiments, the constructs may be a linearized form of DNA, or may be a circularized form of DNA. As such, a construct may comprise a plasmid, a YAC (yeast artificial chromosome), a BAC (bacterial artificial chromosome), or a YAC-BAC hybrid. One or more constructs can be used. A circularized DNA may be linearized within a cell after delivery of the circular construct to a cell. Such constructs can also if desired encode one or more proteins and/or one or more RNA polynucleotides that are used in the described methods. In this regard, as described above, the methods of this disclosure involve the participation of certain proteins. In embodiments, the proteins may be produced within the cell via expression of any suitable expression system that encodes the protein. In embodiments, any protein required to participate in the described process may be modified such that it includes a nuclear localization signal. In embodiments, a protein may be administered directly to the cells. For proteins that require an RNA component to function, such as certain Cas proteins as described herein, the protein(s) and the RNA component may be administered to the cells as ribonucleoproteins (RNPs). Any of the described vectors may also encode a guide-RNA, which may be provided as a single-guide RNA.

As described above, in one embodiment, the MC1 is inserted upstream or downstream of the gene of interest. By using mSwAP-In, the MC1 may be introduced either heterozygously or homozygously, the latter being possible if the gene of interest is on an autosome, and the process repeated using the DNA payload construct with MC2, as shown for example in FIG. 1.

The following description pertains to the functioning of the described mSwAP-In approach. By iteratively switching between two selectable marker cassettes, the system is able to overwrite hundreds of kilobases of mammalian genome segment with synthetic DNA in a complete scarless and iterative manner. In embodiments, from 10 kb-1,000 kb, inclusive, and including all numbers of ranges of numbers there between, are overwritten. In embodiments, at least 100 kb of a mammalian genome is overwritten.

In some embodiments, a cargo sequence (also referred to as a DNA payload, a DNA construct,) may comprise at least 10 kilobases. In some embodiments, a cargo sequence may comprise at least 1, 2, 3, 4, 5, 6, 7, 8, 9, 10, 15, 20, 25, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or at least 1000 kilobases. In some embodiments, a cargo sequence may comprise approximately 1, 2, 3, 4, 5, 6, 7, 8, 9, 10, 15, 20, 25, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or approximately 1000 kilobases.

In some embodiments, a cargo sequence may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 120, 140, 150, 160, 180, 200, 22, 240, 250, 260, 280, 300, 350, 400, 450, 500, 550, 600, 650, 700, 750, 800, 850, 900, 950, or at least 1000 megabases. In some embodiments, a cargo sequence may comprise approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 120, 140, 150, 160, 180, 200, 22, 240, 250, 260, 280, 300, 350, 400, 450, 500, 550, 600, 650, 700, 750, 800, 850, 900, 950, or approximately 1000 megabases.

In some embodiments, a cargo sequence may comprise approximately 350 kilobases. In some embodiments, a cargo sequence may comprise approximately 500 kilobases.

In embodiments, including but not necessarily for use in mice, an endogenous Hprt gene can be knocked out by deploying for example a pair of Cas9-gRNAs targeting the gene (FIG. 1 panel B, step 1) or reversibly knocked out by a floxed stop signal cassette. While embodiments of the disclosure are illustrated using certain recombinases and recombinase recognition sites, alternative recombinases and recombinase recognition sites can be used, and a variety of suitable such components are known in the art. A non-limiting embodiment of this feasibility of this general approach is demonstrated using a similarly reversible knock out in the Piga gene (FIG. 2).

As described above, the synthetic payload DNAs can be assembled in a YAC-BAC shuttle vector, optionally in the following order: UGT-2 kb (or shorter or longer) left homology arm-payload DNA-MC1 or MC2-2 kb (or shorter or longer) right homology arm-UGT (FIG. 1 panel B, step 2 or 3). In this embodiment the payload DNA is co-delivered with a Cas9-gRNA plasmid that recognizes the UGT sequences as well as another Cas9-gRNA plasmid that recognizes the right end boundary (FIG. 1 panel B, step 2 grey scissors), so that the payload DNA can be released in vivo at the same time that the two genomic DNA double-strand breaks are introduced. Alternatively, sequences encoding Cas9 or an alternative guide-directed nuclease and the two gRNA sequences can be combined into a single plasmid, which may improve delivery efficiency. The mSwAP-In genome writing process is designed to be iterable by switching selection from MC1 to selection for MC2 when multiple rounds of mSwAP-In are required (FIG. 1, step 2 and 3). Once the genome writing phase is finished, the final marker cassette can be excised in a final step using either homologous recombination (FIG. 1 panel B, step 4).

The disclosure includes producing a progeny clone that is functionally homozygous; that is, it contains only the synthetic version of the gene and not the native version of the gene, in both chromosomes.

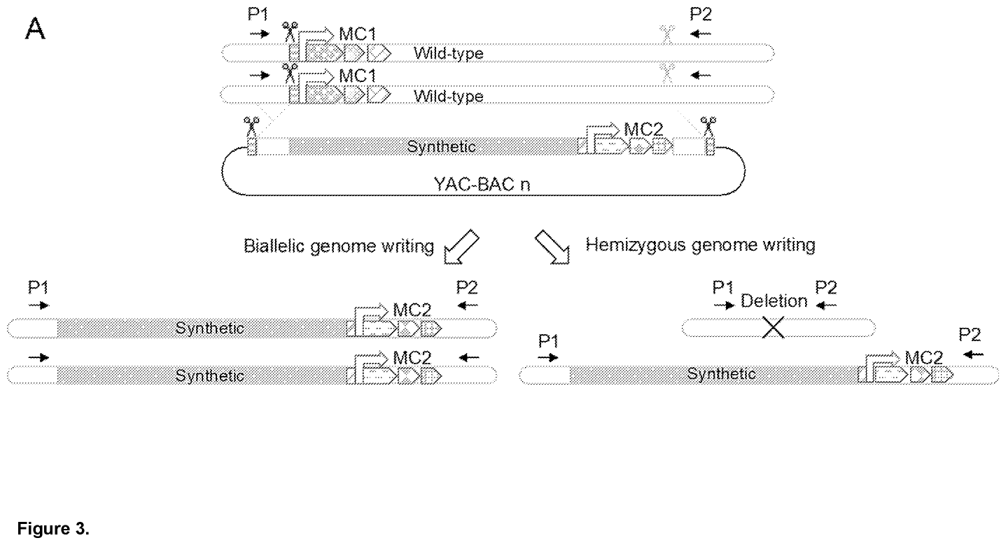

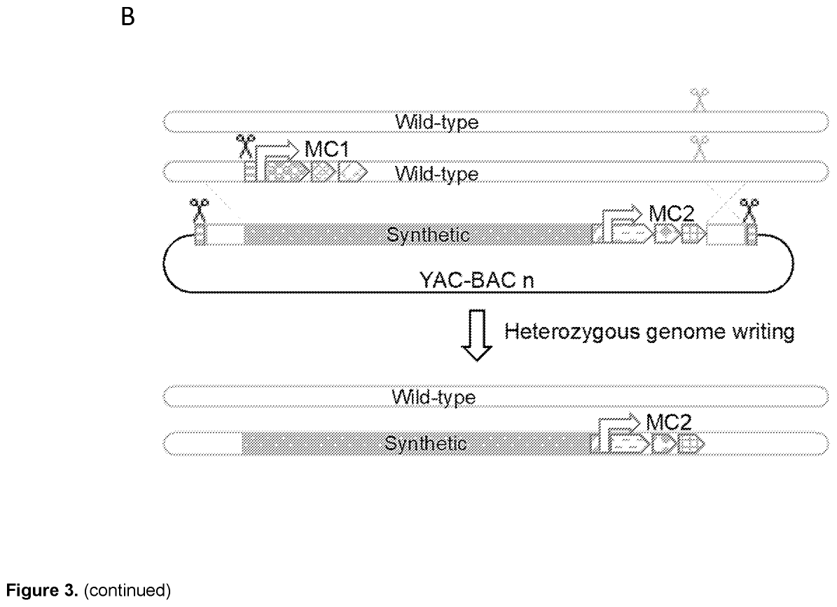

In an embodiment, the disclosure provides for humanizing a gene in, for example, embryonic stem cells (ESCs), such as mouse ESCs (mESCs). This approach comprises producing a mESC in which both murine alleles are deleted and replaced with their human counterparts. This is referred to in the present disclosure as biallelic genome writing. A second approach to produce a functionally homozygous cells is to introduce one synthetic version and to delete the second native allele. This is referred to herein as hemizygous genome writing (FIG. 3 panel A). The third outcome of mSwAP-In is to produce a heterozygous genome, by only inserting MC1 into one of the two alleles and then to only overwrite that single allele with MC1 insertion by mSwAP-In, while retaining one native allele (FIG. 3 panel B).

Delivering a payload with one type of marker cassette can randomly produce either biallelic or hemizygous genome writing clones. To distinguish between clones that are biallelically engineered and clones that are hemizygously engineered, hemizygous clones comprise a deletion of the target locus and thus can be detected using PCR with primer pairs that flank the predicted deletion endpoints (FIG. 3 panel A, P1 and P2). The copy number of the synthetic gene of interest can be determined using quantitative PCR (qPCR) or droplet digital (ddPCR) of the genomic DNA. In addition, the mSwAP-In engineered clones can be subjected to targeted capture sequencing (details described in pubmed.ncbi.nlm.nih.gov/33649239/, the disclosure of which is incorporated herein by reference) of a genomic region of interest, using an appropriate bioinformatic algorithm that detects large-scale deletions can be applied to identify hemizygous clones.

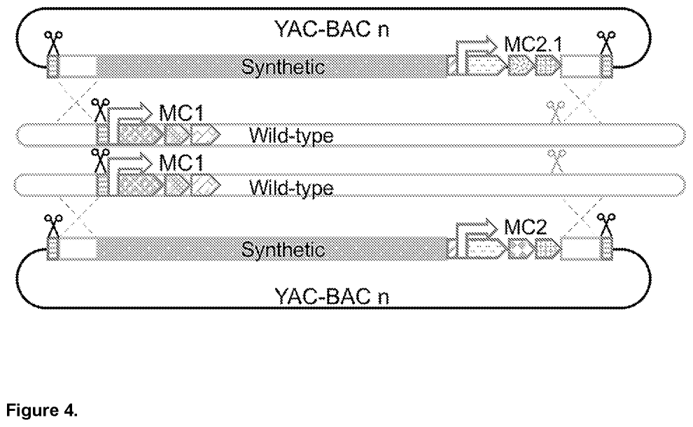

To have enhanced control of producing biallelic genome writing clones after inserting MC1 in both alleles, the disclosure includes concurrent delivery of two versions of payload DNA into the mESCs (or any other type of stem cells). The two versions of payload DNA are otherwise identical, except one comprises a blasticidin (or other selectable marker) resistant gene and the other has neomycin (or other selectable marker) resistant gene. In this disclosure, when the neomycin resistant gene is used in MC2 is referred to as MC2.1. By applying two distinct positive selections and negative selection upon co-delivery, biallelically edited clones will preferentially survive, directly selecting for a biallelically written genomic segment and against heterozygous or hemizygous states (FIG. 4).

The various types of clones that are selected in various stages of mSwAP-In can be screened using a combination of PCR based methods for integration of vector sequences, Cas9 (or other designer nuclease) sequences, and capture sequencing.

As discussed herein, an application of mSwAP-In is making humanized mice in which one or more mouse genes are replaced by their human counterparts, with all of their regulatory regions and introns. The demonstration of producing biallelic or hemizygous humanized mESCs supports making many (e.g., thousands or more) genetically identical mice from those mESCs using tetraploid complementation microinjection approach. Methods for tetraploid complementation are known in the art and can be adapted for making modified mice using the compositions and methods described herein. Suitable approaches are described in, for example, in Nagy et al. 1990 Development (pubmed.ncbi.nlm.nih.gov/2088722/) and Zhao et al. 2010 Nature Protocol (pubmed.ncbi.nlm.nih.gov/20431542/), the disclosures of which are incorporated herein by reference.

The disclosure provides representative and non-limiting reductions to practice of the described compositions and methods. These include four examples of mice derived from ESCs (embryonic stem cells) produced using mSwAP-In. Example 1 is a mouse in which the coronavirus receptor gene Ace2 (an X-chromosomal gene that is naturally “hemizygous” in male cells) is fully humanized using mSwAP-In. Example 2 is a mouse in which the tumor suppressor gene Trp53 (an autosomal gene) and its antisense transcript Wrap53 are both fully humanized on both alleles using mSwAP-In, producing functionally homozygous humanized mice. Example 3 demonstrates serial humanization of an autosomal gene, TMPRSS2, in ACE2 humanized mESCs, demonstrating rapid production of multi-gene humanized GEMM, which I a non-limiting example of a genetically engineered mouse model produced by the described compositions and methods. Example 4 is a mouse in which the native mouse Trp53 tumor suppressor gene is replaced with synthetic copies of that DNA containing designer features, namely a copy of the Trp53 gene that lacks CpG dinucleotides in six p53 mutation hotspots and is therefore believed to be less mutatable than the wild type counterpart.

In embodiments, any sequence in a genome is overwritten. In embodiments, the sequence is overwritten with another sequence which may, as discussed herein, encode a protein. In certain embodiments, the sequence that replaces the endogenous sequence includes a correction of a mutation. In embodiments, the mutation is a mutation that is associated with cancer or another genetic disorder. In embodiments, the replaced sequence comprises a p53 mutation, a mutation of any kinase, such as any mutated tumor suppressor gene, a mutated KRAS, a mutated Bruton's tyrosine kinase (BTK), a mutated version of any member of the epidermal growth factor receptor (EGFR) family, and the like. In embodiments, the replaced sequence comprises a segment of a chromosome that is associated with a monoallelic mutation that is correlated with a disorder. In embodiments, the replaced sequence comprises a segment of a chromosome that is correlated with an indel, such as certain forms of muscular dystrophy.

In embodiments, the replaced sequence comprises an Ace2 gene, a Trp53 gene, a Wrap antisense transcript, a Tmprss2 gene, or a combination thereof. In embodiments, the replaced sequence comprises a sequence that encodes any enzyme, an intracellular or cell surface receptor, a growth factor, an antibody or antigen binding segment of an antibody, a checkpoint inhibitor ligand, or a protein prodrug. In embodiments, the replaced sequence comprises a sequence that encodes a protein that can be secreted from cells that have been modified by using the described compositions and methods.

### Other Embodiments

Embodiment 1. A composition comprising a first contiguous DNA molecule comprising:
(i) a first homology segment that is homologous to a first segment of a chromosome;
(ii) a first marker cassette (MC1) comprising a first guide RNA (gRNA) target site (UGT1);
(iii) a second homology segment that is homologous to a second segment of the chromosome;
wherein the first gRNA target site is not endogenous to the genome of a cell prior to introducing the MC1 to the cell.
Embodiment 2. A composition comprising a first contiguous DNA molecule comprising:
(i) a first homology segment that is homologous to a first segment of a chromosome;
(ii) a first marker cassette (MC1) comprising a first guide RNA (gRNA) target site (UGT1);
(iii) a second homology segment that is homologous to a second segment of the chromosome;
wherein the first gRNA target site is not endogenous to the genome of a cell prior to introducing the MC1 to the cell; and
a second contiguous DNA molecule comprising:
(i) a copy of the first gRNA target site;
(ii) the first homology segment;
(iii) a first heterologous DNA payload; and
a second marker cassette (MC2) comprising;
(i) a second gRNA target site (UGT2) that is different than the first gRNA target site; and
a third homology segment.
Embodiment 3. The composition of embodiment 2, wherein the composition further comprises a third contiguous DNA molecule comprising in a 5′-3′ direction:
(i) a copy of the UGT2;
(ii) a fourth homology segment;
(iii) a second heterologous DNA payload;
(iv) a copy of the MC1
(v) a fifth homology segment; and
(vi) a second copy of the UGT2.
Embodiment 4. The composition of embodiment 2 or 3, wherein the composition is introduced into a cell.
Embodiment 5. The composition of embodiment 4, wherein the cell is a mammalian cell.
Embodiment 6. The composition embodiment 4 or 5, wherein the composition is introduced into an endogenous chromosome segment of the cell.
Embodiment 7. The composition of any one of embodiments 4-6, wherein UGT1 and/or UGT2 is not endogenous to the endogenous chromosome segment of the cell prior to introduction of the composition into the cell.
Embodiment 8. The composition of any one of the preceding embodiments, wherein the composition further comprises a UGT1 independent nuclease that cleaves a segment of the chromosome that is upstream of and not present in the third homology segment or the DNA payload.
Embodiment 9. The composition of any one of the preceding embodiments, wherein the composition further comprises a UGT1 gRNA-directed nuclease.
Embodiment 10. The composition of any one of the preceding embodiments, wherein introduction of the UGT1 gRNA-directed nuclease promotes homologous recombination based replacement of the entire MC1 via the first homology segment and the third homology segment.
Embodiment 11. The composition of any one of the preceding embodiments, wherein MC1 comprises a promoter, a first reporter protein, a first positive selection marker, and/or a first negative selection marker.
Embodiment 12. The composition of any one of the preceding embodiments, wherein MC2 comprises a promoter, a second reporter protein, a second negative selection marker, and/or a second positive selection marker.
Embodiment 13. The composition of any one of the preceding embodiments, wherein the first reporter protein, the first positive selection marker and/or the first negative selection marker of MC1 is different than the second reporter protein, the second positive selection marker, and/or the second negative selection marker of MC2.
Embodiment 14. The composition of any one of the preceding embodiments, wherein the promoter of MC1 and/or MC2 are the same promoter.
Embodiment 15. The composition of any one of the preceding embodiments, wherein the first reporter protein, the first positive selection marker and/or the first negative selection marker of MC1 is the same as the second reporter protein, the second positive selection marker, and/or the second negative selection marker of MC2.
Embodiment 16. The composition of any one of the preceding embodiments, wherein the first, and/or second reporter protein comprises a fluorescent protein.
Embodiment 17. The composition of any one of the preceding embodiments, wherein the first and/or second positive selection marker comprises an antibiotic resistance gene.
Embodiment 18. The composition of embodiment 17, wherein the antibiotic resistance gene comprises N-acetyltransferase (pac), Blasticidin S deaminase (bsd), Neomycin (G418) resistance gene (neo), Hygromycin resistance gene (hygB), Zeocin resistance gene (Sh ble), or hypoxanthine phosphoribosyltransferase 1 gene (HPRT1).
Embodiment 19. The composition of any one of the preceding embodiments, wherein the first and/or second negative selection marker comprises a toxin-antitoxin system, or a counter selection.
Embodiment 20. The composition of any one of the preceding embodiments, wherein the first and/or second negative selection marker comprises herpes simplex virus type 1-thymidine kinase (HSV1-TK), human HPRT1, or the cytosine deaminase gene (codA).
Embodiment 21. The composition of any one of the preceding embodiments, wherein the first homology segment is upstream of the endogenous gene to be replaced and the second homology segment is downstream of the endogenous gene to be replaced.
Embodiment 22. The composition of any one of embodiments 1-20, wherein the first homology segment is downstream of the endogenous gene to be replaced and the second homology segment is upstream of the endogenous gene to be replaced.
Embodiment 23. The composition of any one of the preceding embodiments, wherein the cell comprising the first heterologous DNA payload and MC2 is selected using the second positive and first negative selection based on the second positive (MC2) and first (MC1) negative selection markers and optionally one or both reporter proteins.
Embodiment 24. The composition of any one of the preceding embodiments, wherein the cell comprising the second heterologous DNA payload and MC1 is selected using the first positive (MC1) and second negative (MC2) selection based on the first positive and second negative selection markers.
Embodiment 25. The composition of any one of the preceding embodiments, wherein the heterologous DNA payload is only inserted into one homologous chromosome of the cell to thereby provide a heterozygous or hemizygous chromosome pair in which only one chromosome in the pair comprises the heterologous DNA payload.
Embodiment 26. The composition of any one of the preceding embodiments, wherein the heterologous DNA payload is inserted into both homologous chromosomes of the cell to thereby provide a homozygous chromosome pair in which both chromosomes in the pair comprises the heterologous DNA payload.
Embodiment 27. The composition of any one of the preceding embodiments, wherein the heterologous DNA payload comprises a gene.
Embodiment 28. The composition of any one of the preceding embodiments, wherein the cell comprises a human cell, or a non-human cell.
Embodiment 29. The composition of any one of the preceding embodiments, wherein the cell comprises a totipotent stem cell, a pluripotent stem cell, a multipotent stem cell, or an oligopotent stem cell.
Embodiment 30. The composition of embodiment 28, wherein the non-human cell comprises a mouse stem cell which is optionally a mouse embryonic stem cell.
Embodiment 31. The composition of embodiment 30, wherein the mouse embryonic stem cell is diploid (2n) and is optionally present in or is introduced into a tetraploid (4n) mouse blastocyst.
Embodiment 32. A pseudopregnant mouse comprising the blastocyst of embodiment 31.
Embodiment 33. A mouse obtained from the pseudopregnant mouse of embodiment 32, and/or a progeny of the pseudopregnant mouse.
Embodiment 34. An isolated DNA comprising the recombinant polynucleic acids encoding MC1 and/or MC2 of any one of the preceding embodiments.
Embodiment 35. An expression vector encoding MC1 and/or MC2 of any one of the preceding embodiments.
Embodiment 36. A kit comprising a DNA molecule comprising an MC1 for embodiment 1 or 2, or an MC2 of embodiment 2, or a combination of DNA molecules comprising the MC1 and the MC2.
Embodiment 37. The kit of embodiment 36, further comprising a guide RNA or a vector encoding the guide RNA, said guide RNA being targeted to a UGT1 or UGT2, or a combination of said guide RNAs, or one or more vectors encoding the combination of guide RNAs.
Embodiment 38. The kit of embodiment 36 or 37, further comprising a nuclease, or a vector encoding the nuclease, wherein the nuclease is specific for the MC1 or the MC2, or a combination of said nucleases.
Embodiment 39. The kit of embodiment 38, further comprising at least one agent that can be used for selection based on the positive or negative selection markers, or a combination of said agents.
Embodiment 40. A mouse obtained from the pseudopregnant mouse of embodiment 32 and/or a progeny of the pseudopregnant mouse, wherein the first or the second payload encodes an angiotensin converting enzyme 2 (ACE2).
Embodiment 41. The mouse of embodiment 40, wherein the ACE2 is a human ACE2.
Embodiment 42. The heterologous DNA payload of any one of embodiments 2-41, wherein the payload is a human angiotensin converting enzyme 2 (ACE2).
Embodiment 43. A pharmaceutical composition comprising the composition of any one of embodiments 1-31, and a pharmaceutically acceptable excipient or carrier.
Embodiment 44. A method of treating a disease in a subject in need thereof, comprising administering a therapeutically effective amount of the pharmaceutical composition of embodiment 43.
Embodiment 45. A method of modifying a chromosome in a mammalian cell to replace an endogenous chromosome segment with a heterologous chromosome segment, comprising administering the composition of any one of embodiments 1-31 to the cell.
Embodiment 46. The method of embodiment 45, further comprising iteratively administering additional heterologous DNA payloads to the cell to replace the endogenous chromosome segment, wherein the additional heterologous DNA payloads comprise MC1 if MC2 was administered previously, and/or MC2 if MC1 was administered previously.
Embodiment 47. A method for modifying a chromosome in a mammalian cell to replace an endogenous chromosome segment with a heterologous chromosome segment, wherein the heterologous chromosome segment comprises a DNA payload, the method comprising: (A) introducing into the mammalian cell a first contiguous DNA molecule comprising: (i) a first homology segment that is homologous to the endogenous chromosome segment that is either upstream or downstream of an endogenous sequence to be replaced; (ii) a first marker cassette (MC1) comprising: (a) a first guide RNA (gRNA) target site that is not endogenous to the genome of the mammalian cell prior to introducing the MC1, wherein the first gRNA target site is a universal gRNA target sequence (UGT1); b) a promoter; (c) a sequence encoding a first reporter protein, the first reporter protein optionally comprising a fluorescent protein; (d) a sequence encoding a first positive selection marker; and (e) a sequence encoding a first negative selection marker; (iii) the first contiguous DNA molecule further comprising a second homology segment that is homologous to a contiguous the endogenous chromosome in (A) (i) that is downstream of the endogenous sequence to be replaced if (A) (i) is upstream and is upstream of the endogenous sequence to be replaced if (A) (i) is downstream; (B) using a site specific nuclease recognizing a sequence endogenous to the mammalian cell and independent of the UGT1, to promote homologous recombination of the entire MC1 via the first homology segment and the second homology segment; and selecting a mammalian cell that comprises the MC1 using positive selection based on the first positive selection marker, and optionally the first reporter protein, to create a selected mammalian cell.
Embodiment 48. The method of Embodiment 47, further comprising: (C) introducing into the selected mammalian cell which comprises the MC1 a second contiguous DNA molecule comprising: g) a copy of the UGT1; h) the first homology segment of (A) (i); i) a first heterologous DNA payload for replacement of an upstream segment of a first segment of the endogenous sequence, the first DNA payload optionally comprising a regulatory element operably linked to a heterologous gene; and a second marker cassette (MC2) comprising: j) a second gRNA target site that is not endogenous to the genome of the one or more mammalian cells, wherein the second gRNA target site is a universal gRNA target sequence 2 (UGT2) that has a sequence that is different from the sequence of UGT1; k) a promoter; 1) a sequence encoding a second reporter protein, the second reporter protein optionally comprising a fluorescent protein; m) a sequence encoding a second negative selection marker; and n) a sequence encoding a second positive selection marker; the second contiguous DNA molecule comprising a third homology segment that is homologous to a segment of the gene that is downstream of the MC1; o) a copy of the UGT1; (D) using a UGT1 independent nuclease to cleave a segment of the chromosome that is upstream of and not present in the third homology segment or the payload, and using a UGT1 gRNA-directed nuclease, to promote homologous recombination based replacement of the entire MC1 via the first homology segment and the third homology segment, and selecting a mammalian cell that comprises the first heterologous DNA payload and MC2 using the second positive and first negative selection based on the second positive (MC2) and first (MC1) negative selection markers and optionally one or both reporter proteins.
Embodiment 49. The method of Embodiment 48, further comprising: (E) introducing into the mammalian cell selected in (D) a third contiguous DNA molecule comprising in a 5′-3′ direction: p) a copy of the UGT2; q) a fourth homology segment that is homologous to a segment that is immediately upstream of MC2; r) a second heterologous payload coding sequence for replacement of a segment of the endogenous sequence that is immediately downstream of the first heterologous sequence; s) a copy of MC1; t) a fifth homology segment that is homologous to a segment of the chromosome that is downstream of the MC2; and u) a copy of the UGT2; (F) using a UGT2 independent nuclease to cleave a segment of the chromosome that is upstream of and not present in the fifth homology segment or the payload, and using a UGT2 guide RNA-directed nuclease to promote homologous recombination replacement of the entire MC2 via the fourth and fifth homology segments so that the chromosome comprises the second heterologous payload and MC1, and selecting mammalian cells that comprise the second heterologous payload and MC1 using the first positive (MC1) and second negative (MC2) selection based on the first positive and second negative selection markers, and optionally removing the MC1.
Embodiment 50. The method of embodiment 47 or 48, wherein the DNA payload is only inserted into one homologous chromosome to thereby provide a heterozygous or hemizygous chromosome pair in which only one chromosome in the pair comprises the DNA payload.
Embodiment 51. The method of embodiment 47 or 48, wherein the DNA payload is inserted into both homologous chromosomes to thereby provide a homozygous chromosome pair in which both chromosomes in the pair comprises the DNA payload.
Embodiment 52. The method of any one of embodiments 47-51, wherein the DNA payload comprises a gene.
Embodiment 53. The method of any one of embodiments 47-52, wherein the one or more mammalian cells comprise human cells, or non-human mammalian cells.
Embodiment 54. The method of embodiment 53, wherein the one or more mammalian cells comprise totipotent, pluripotent, multipotent, or oligopotent stem cells.
Embodiment 55. The method of embodiment 53 or 54, wherein the non-human mammalian cells comprise mouse stem cells which are optionally mouse embryonic stem cells.
Embodiment 56. The method of embodiment 55, wherein the mouse embryonic stem cells are diploid (2n) and are optionally present in or are introduced into a tetraploid (4n) mouse blastocyst.
Embodiment 57. A pseudopregnant mouse comprising a blastocyst of embodiment 56.
Embodiment 58. A mouse obtained from the pseudopregnant mouse of embodiment 57, and/or progenety of said mouse.
Embodiment 59. An isolated DNA comprising sequences encoding MC1 or an MC2 of embodiment 47.
Embodiment 60. An expression vector encoding an MC1 or an MC2 of embodiment 47.
Embodiment 61. A kit comprising a DNA molecule comprising an MC1 or an MC2 of embodiment 47, or a combination of DNA molecules comprising the MC1 and the MC2.
Embodiment 62. The kit of embodiment 61, further comprising a guide RNA or a vector encoding the guide RNA, said guide RNA being targeted to a UGT1 or UGT2, or a combination of said guide RNAs, or one or more vectors encoding the combination of guide RNAs.
Embodiment 63. The kit of embodiment 61 or 62, further comprising a nuclease, or a vector encoding the nuclease, wherein the nuclease is specific for the MC1 or the MC2, or a combination of said nucleases.
Embodiment 64. The kit of embodiment 63, further comprising at least one agent that can be used for selection based on the positive or negative selection markers, or a combination of said agents.

### EXAMPLES

The following Examples are intended to illustrate but not limit the disclosure.

### Example 1: Humanizing an X-Linked Gene, ACE2, in Mouse/Rapid Engineering of an ACE2 Fully Humanized Mouse Model Using mSwAP-In

ACE2 encodes the receptor for SARS-COV-2 coronavirus which causes COVID-19. The mouse counterpart does not bind to the SARS-COV-2 spike protein and as a result the mice do not get infected by the virus. A simple constitutive transgene expressing human ACE2 protein in all tissues is susceptible to the virus, but the mice succumb to infection within days—a phenotype not observed in humans. We therefore designed and engineered a fully humanized A (E2 mouse model with both upstream and downstream regulatory DNA elements as well as all the intronic elements of human ACE2 gene in a mouse genetic context.

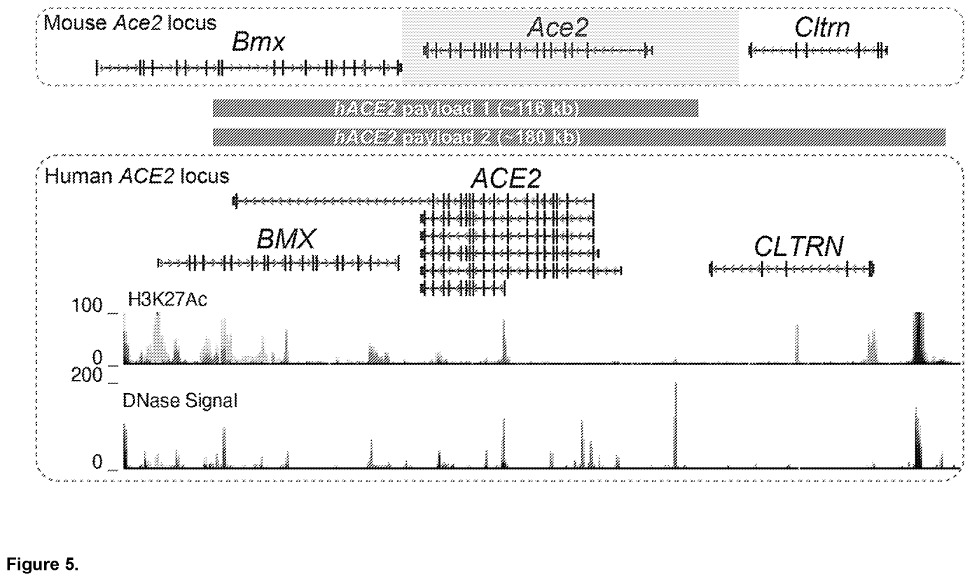

For the human ACE2 gene (hACE2), we identified a predicted long transcript variant (NM_001386259.1) that spans 82,764 bp in the genome and overlaps with the BMX gene (FIG. 5). In contrast to the canonical transcript that produces an 805-aa length protein, the long transcript produces a 786-aa length protein. This long transcript has not been well-characterized, and may have important biological functions. Consequently, to avoid losing any biological information in our ACE2 humanized mESC line, we defined the left boundary of our construct to include this long transcript of ACE2 (chrX: 15,510,303, hg38). For the right boundary, by checking the epigenetic hallmarks such as DNase hypersensitive sites and H3K27 acetylation sites that indicate the functional DNA elements in the UCSC genome browser, we observed a DNase signal peak downstream of the CLTRN gene which might represent an enhancer of the ACE2 gene. We designed a short version (116 kb) of the ACE2 payload spanning from the left boundary to the 3′ end of the CLTRN gene (chrX: 15,510,303-15,625,823, hg38). Also, we identified a strong DNase and H3K27Ac signal upstream of A (097625.2, which encodes the angiotensin converting enzyme-like protein that serves as the receptor for coronaviruses SARS-COV-1 and HCoV-NL63. To test whether A (097625.2 also serves as the receptor for SARS-COV-2, we designed a long version (180 kb) of the ACE2 payload spanning from the left boundary to the 5′ end of A (097625.2 (chrX: 15,510,303-15,690,440, hg38) (FIG. 5).

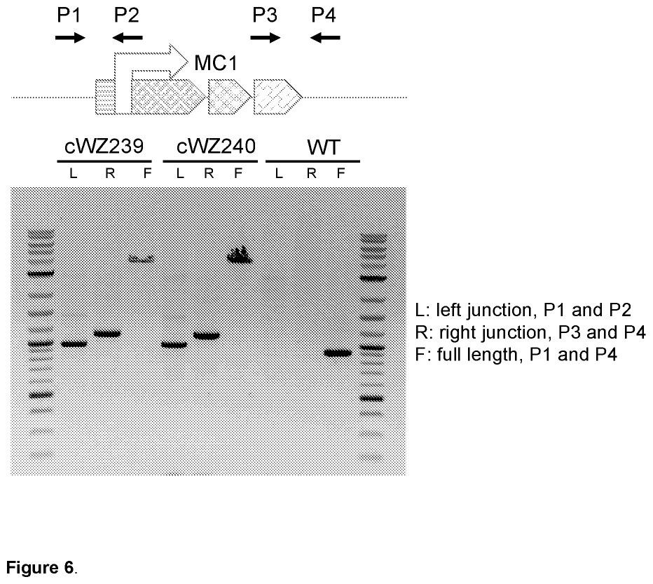

To overwrite the mouse Ace2 (mAce2) locus with its human counterpart, we first inserted the marker cassette 1 downstream of Ace2 in a male C57BL/6J mouse embryonic stem cell line. The insertion of marker cassette 1 was confirmed by junction PCR (FIG. 6) and Sanger sequencing (data not shown).

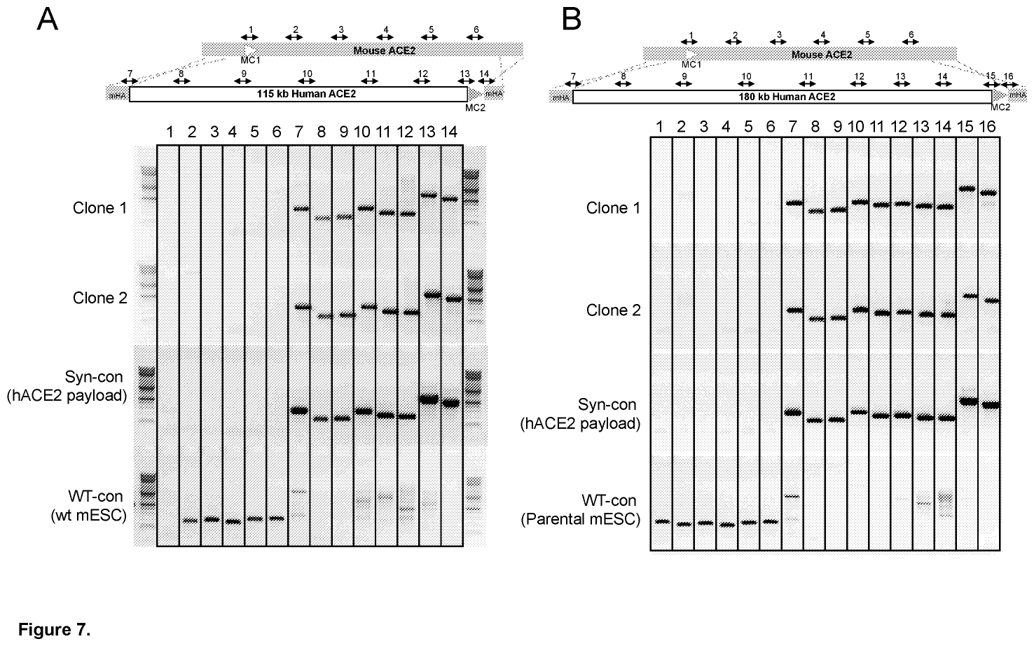

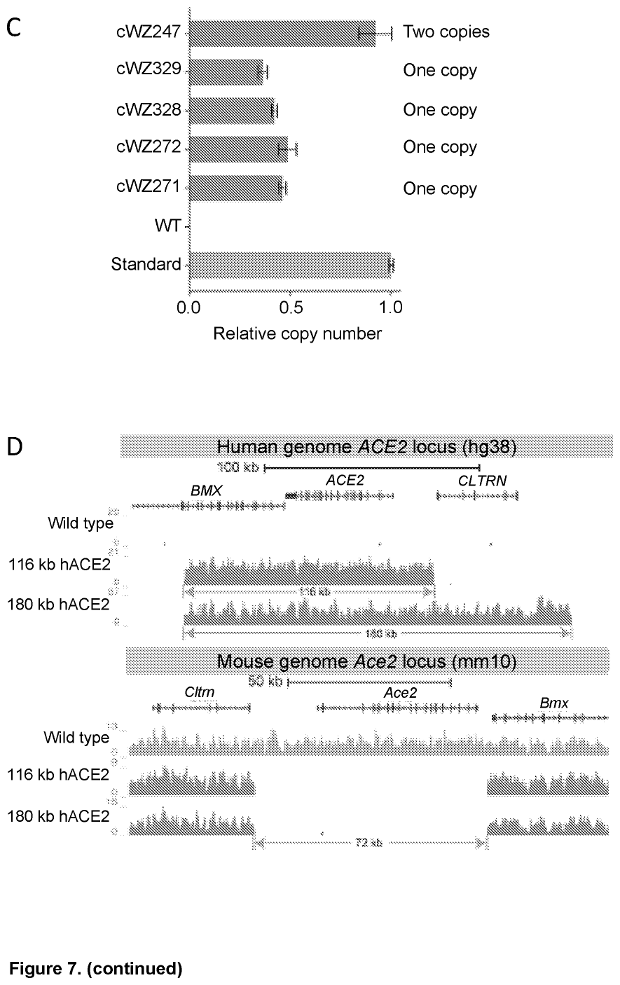

Next, we delivered both the 116 kb hACE2 and 180 kb hACE2 payloads into MC1 founder line using mSwAP-In. We used feeder-dependent cell culture conditions to maintain the developmental potential of the mESCs, while splitting cells from each clone into a feeder-independent subculture for genotyping and sequencing. The mESC clones showed the expected “fluorescence marker switch”, indicating the swap of marker cassette along with payload DNAs (data not shown). To ensure the mAce2 locus was fully overwritten by the two hACE2 payloads, we performed genotyping PCR using multiple primer pairs across mAce2 and hACE2 regions. Correct clones showed the presence of hACE2 amplicons and the absence of mAce2 amplicons (FIG. 7 panels A and B). The overall efficiency was 61.5% (n=13) for the 116 kb hACE2 payload, and 60.8% (n=79) for the 180 kb hACE2 payload as determined by genotyping PCR. To enable hACE2 copy number quantification, we constructed a plasmid containing one copy of mActb and one copy of hACE2 to serve as a standard in qPCR analysis, and identified mESC clones with one copy of hACE2 (FIG. 7 panel C). Lastly, Capture-seq analysis verified that the hACE2 mESC clones have even coverage across the ACE2 region with no deletion or duplication events, as well as the loss of mouse Ace2 (FIG. 7 panel D). We also confirmed a lack of off-target integration for hACE2 by bamintersect analysis, and no Cas9 reads were captured in these mESC clones.

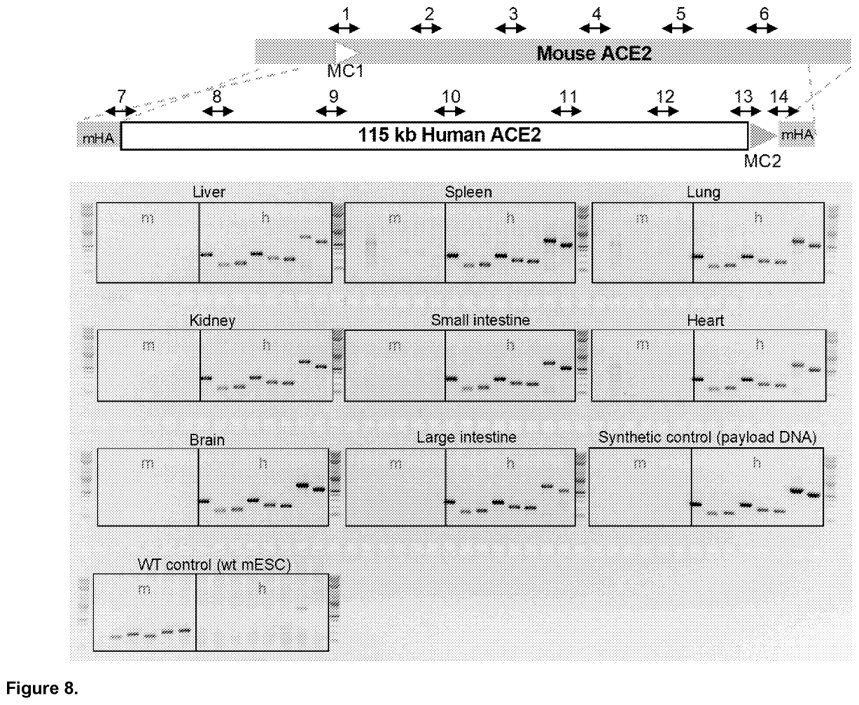

Mouse ES clones with mouse Ace2 replaced by human ACE2 were injected into tetraploid blastocyst embryos for mouse production. 22 pups were produced from two 116 kb ACE2 mES clones. We genotyped various tissues from a tetraploid complementation-derived mouse, and detected only hACE2 amplicons (FIG. 8).

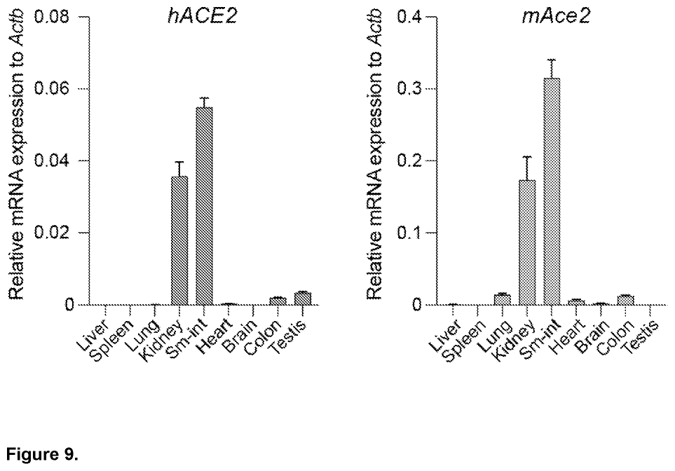

To check the expression pattern of human ACE2 in our fully humanized mouse model, we examined hACE2 mRNA expression across nine tissues from the 116 kb hACE2 mouse. Abundant hACE2 mRNA was detected in small intestine and kidney, while moderate levels were observed in testis and colon, indicating the mouse transcription machinery faithfully expressed hACE2. Overall, expression patterns between mAce2 and hACE2 were similar aside from a few important human-specific differences. For instance, we readily detected hACE2 in the testis, recapitulating the ACE2 expression observed in humans, whereas mAce2 is not expressed in testis of wild-type mice. In addition, we observed lower hACE2 expression in the lung of the hACE2 mice compared with mAce2 in wild-type mice (FIG. 9), consistent with the comparison between human and mouse RNA-Seq data.

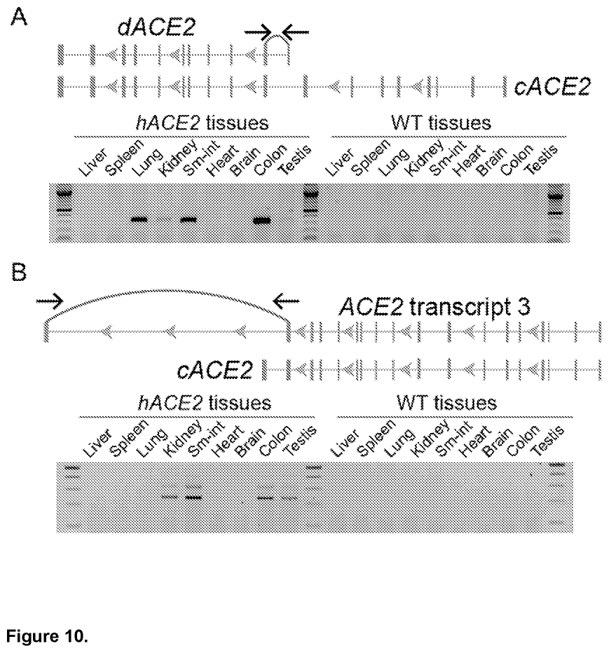

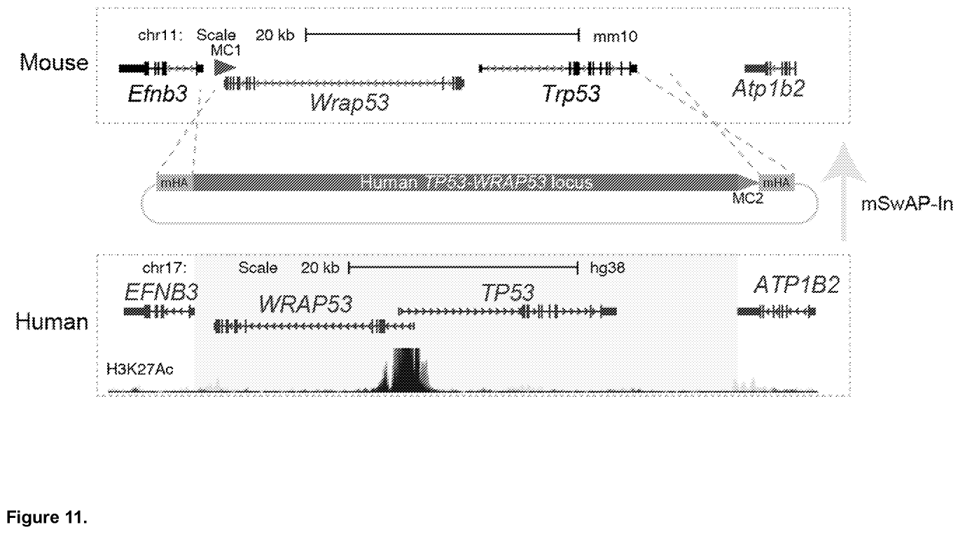

To check whether human-specific splicing patterns would be recapitulated in the hACE2 mice we performed RT-PCR assay and readily detected both a novel ACE2 transcript 5, dACE2, and the long ACE2 transcript 3. These data further demonstrate that physiological alternative splicing patterns of human ACE2 are recapitulated in the hACE2 mice (FIG. 10).

TABLE 1
Primer sequences used in this Example

| | Forward | Reverse |
|---|---|---|
| Mouse | GATCAAGATCATTGCTCCTCC | AAGGGTGTAAAACGCAGCTCA |
| Actb | TGA (SEQ ID NO: 1) | (SEQ ID NO: 2) |
| Mouse | TGATGAATCAGGGCTGGGATG | ATTCTGAAGTCTCCGTGTCCC |
| Ace2 | (SEQ ID NO: 3) | (SEQ ID NO: 4) |
| Human | GGGATCAGAGATCGGAAGAAG | AGGAGGTCTGAACATCATCAG |
| ACE2 | AAA (SEQ ID NO: 5 | TG (SEQ ID NO: 6) |

### Example 2: Humanizing an Autosomal Gene, TP53 Shows Direct Production Via Hemizygous or Biallelic Replacement Using mSwAP-In and Rapid Production of the Derived Mice by Tetraploid Complementation

Genomically humanized mouse models provide platforms for disease modeling and therapeutic development. The tumor suppressor p53 plays vital roles in growth arrest, DNA repair, senescence and apoptosis in cells. Numerous studies have shown that over half of cancer genomes contain p53 missense mutations, emphasizing the importance of p53 mutations in cancer. The existing human p53 knock-in (hupki) mouse model is the predominant humanized p53 murine model, in which substitution of endogenous mouse Trp53 (mTrp53) exons 4-9 with human TP53 (hTP53) exons 4-9. This mouse-human chimeric p53 mouse model is useful when studies focus on mutagenesis of the p53 DNA binding domain, but human p53 exons other than 4-9 are lacking in this model, and it cannot recapitulate transcriptional and posttranscriptional regulation of the full length human TP53 gene, nor the human specific p53 isoforms produced by alternative splicing. We utilized biallelic mSwAP-In approach to construct a fully humanized p53 mouse model by replacing the entire mouse Trp53-Wrap53 genes with their human counterparts, and hope to evaluate the regulation of human p53 in a mouse context (FIG. 11).

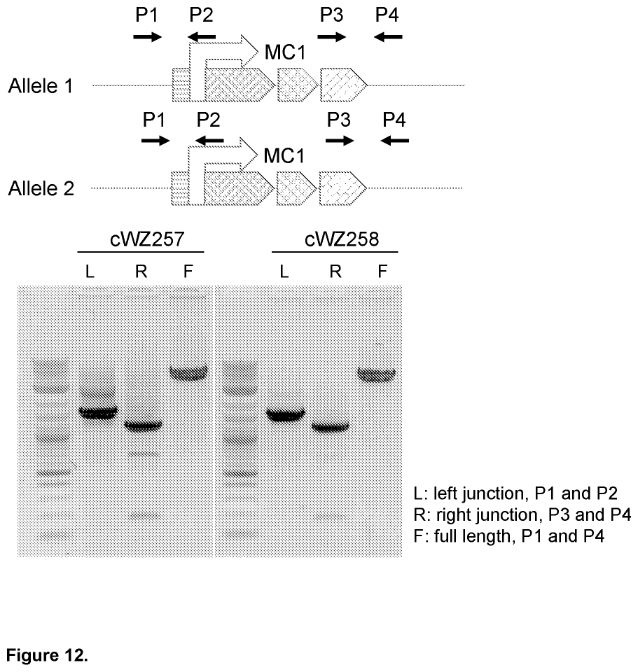

Following the mSwAP-In procedure (FIG. 1), the first step is to insert marker cassette 1 at a location just downstream of mouse Trp53. We experimentally concluded that Homology Directed Repair without in vivo linearization produced the highest knock-in efficiency and minimal number of background colonies. MC1 was inserted biallelically downstream of the mouse Trp53 gene, and desired clones were verified by junction PCR (FIG. 12).

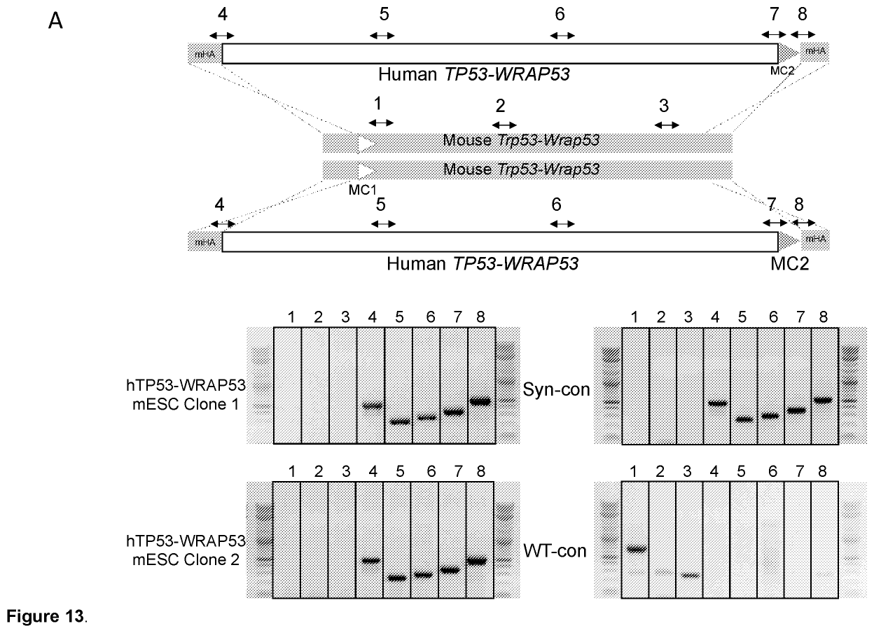

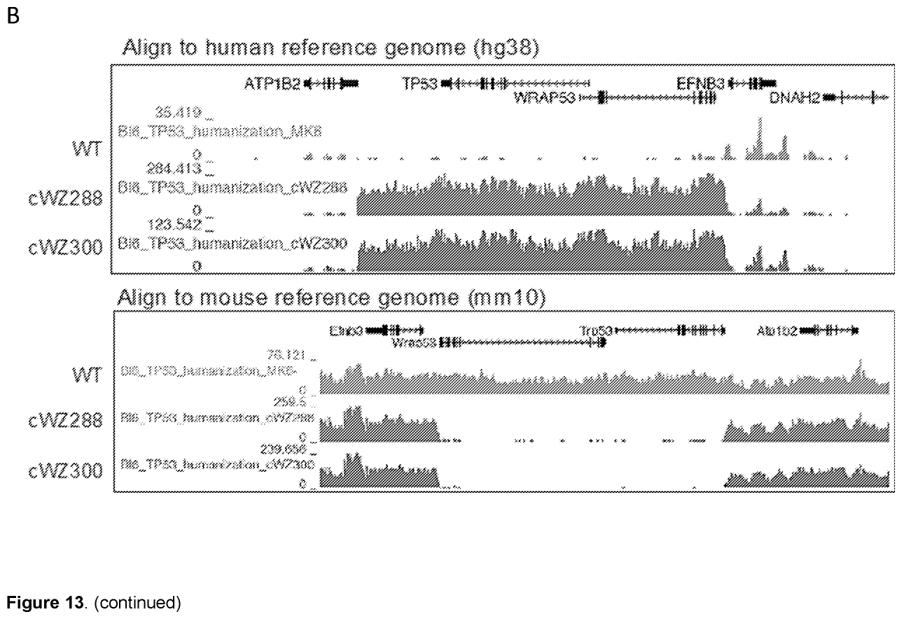

After sequentially applying positive (blasticidin) and negative (ganciclovir) selections, mESC clones were genotyped using natural “watermarks” based on sequence differences between the mouse and human genes (FIG. 13 panel A). Capture sequencing result confirms the genotyping PCR conclusion (FIG. 13 panel B), the overall efficiency was 17.3% (n=98).

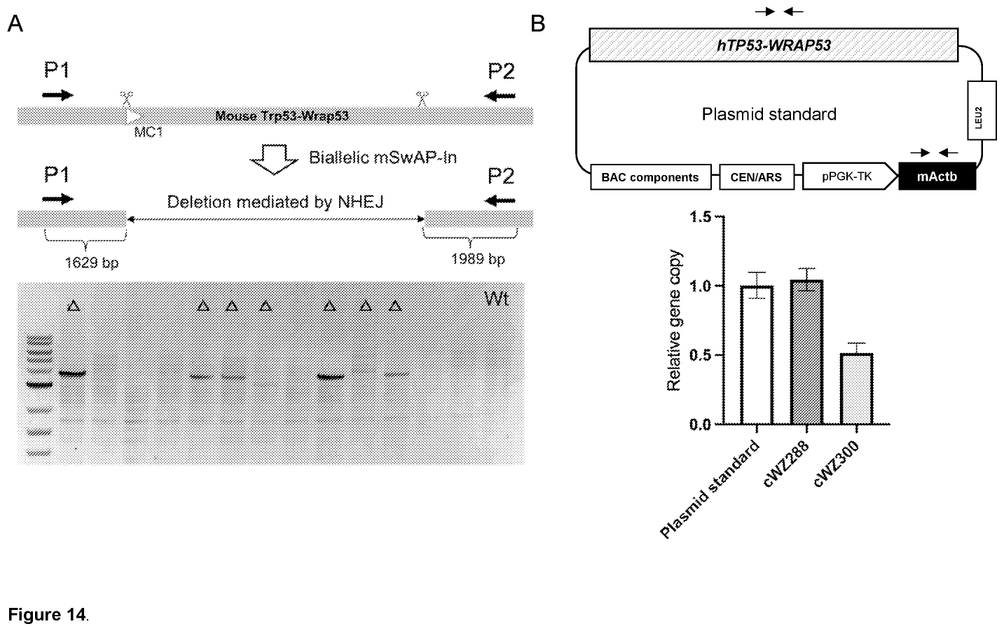

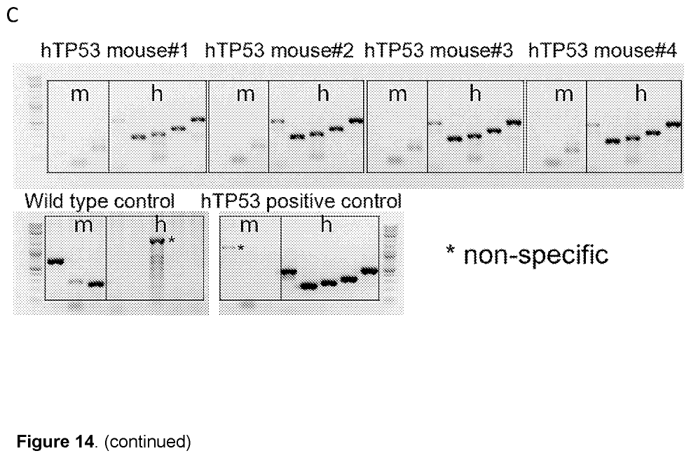

As discussed above, we found biallelic mSwAP-In could also produce hemizygous clones (in which one allele undergoes a large segment deletion of the native mouse allele mediated by Non-Homologous End Joining), which can be readily detected by using primers adjacent to two DNA break points (FIG. 14 panel A, P1 and P2). Of the 13 TP53-WRAP53 humanized clones we tested, 7 of them were shown to be hemizygous by this assay (FIG. 14 panel A). To definitively determine the human TP53-WRAP53 copy number delivered in mESCs, a plasmid DNA containing one copy of human TP53-WRAP53 gene cassette and one copy of the mouse Actb gene was constructed. Using the plasmid as control, the copy number of TP53-WRAP53 relative to Actb can be readily determined by a qPCR assay (FIG. 14 panel B). Biallelically humanized TP53-WRAP53 mESCs were used to generate mice using tetraploid complementation. A total of 28 pups were obtained after injecting and implanting 124 tetraploid embryos. Representative mouse biopsy PCR results are shown in FIG. 14 panel C.

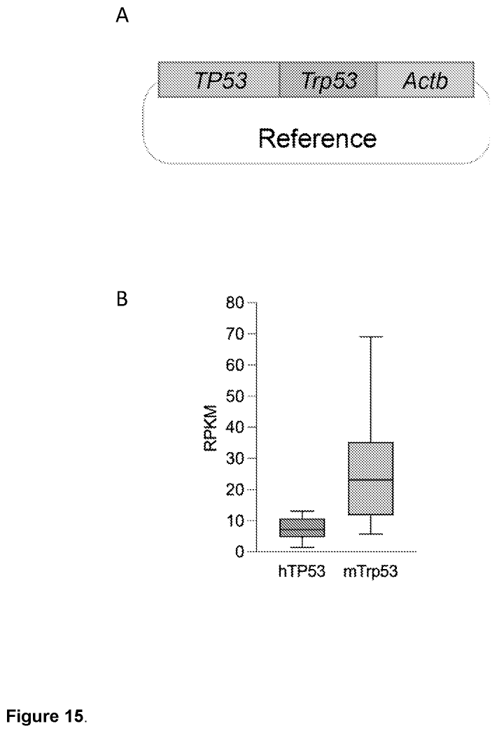

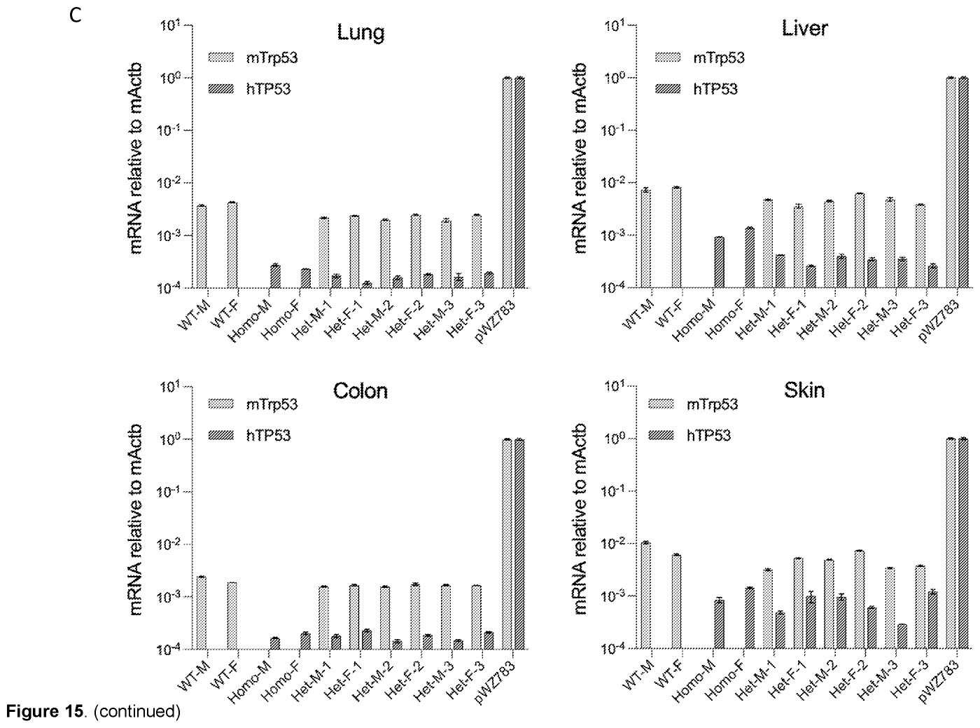

To test whether human TP53 is faithfully expressed in mouse, also understand the expression level of human TP53 relative to mouse Trp53, we examined hTP53 and mTrp53 expression in lung, liver, colon and skin of both homozygous and heterozygous TP53-WRAP53 humanized mice via RT-qPCR. We first constructed a reference DNA containing the qPCR region from hTP53, mTrp53 and mActb for normalization purpose (FIG. 15 panel A). We also gathered publicly available information about hTP53 and mTrp53 expression levels in human and mouse respectively, showing that hTP53 has a ˜3 fold lower RPKM RNA levels compared to mTrp53 (FIG. 15B). RT-qPCR result showed that hTP53 expresses in all four tissues, with a 3˜10 fold lower expression level relative to mTrp53 (FIG. 15 panel C).

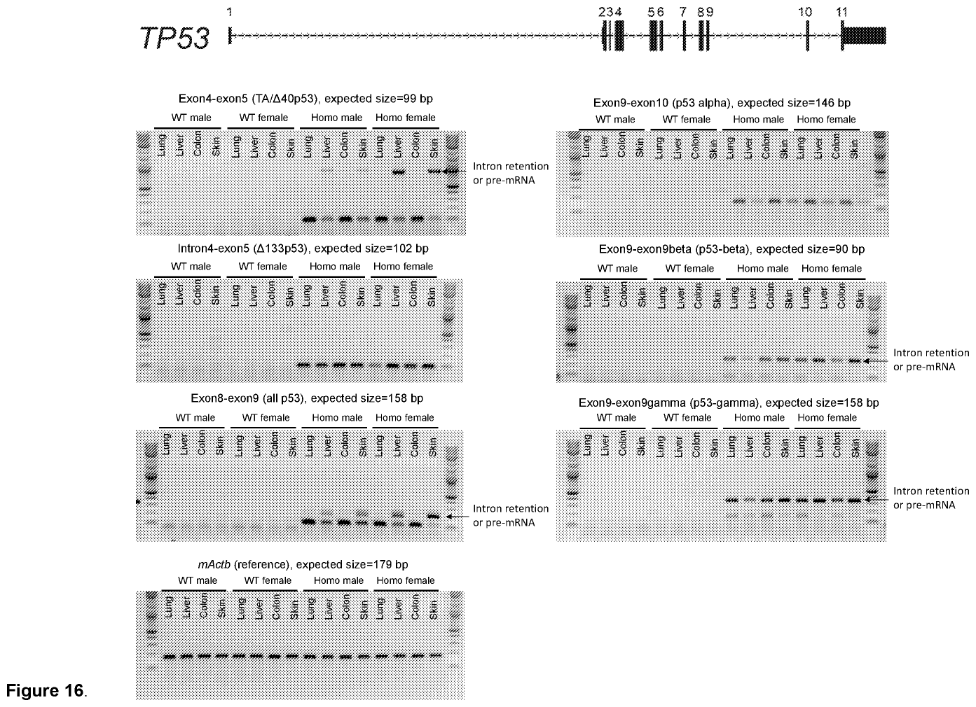

p53 isoform expressions play important roles in clinical outcome of cancer patients. Next we used a panel of primers to detect the key p53 isoforms (Δ40p53, Δ133p53, p53α, p53β, p53γ) produced in human. RT-PCR results show all five isoforms were detected only in TP53-WRAP53 humanized lung, liver, colon and skin, but not in those of wild-type mice (FIG. 16).

TABLE 2
Primers used for p53 isoforms detection.

| p53 | Primer | Targeted | |
|---|---|---|---|
| isoforms | name | region | 5′-3′ sequence |
| TA/Δ40p53 | (F) | Exon 4 | CAGCCAAGTCTGTGACTTGCA (SEQ ID NO: 7) |
| | (R) | Exon 5 | GTGTGGAATCAACCCACAGCT (SEQ ID NO: 8) |
| Δ133p53 | (F) | Intron 4 | ACTCTGTCTCCTTCCTCTTCCTACAG (SEQ ID NO: 9) |
| | (R) | Exon 5 | Same as TAp53 (R) |
| α (alpha) | (F) | Exon 9 | CACTGCCCAACAACACCAGCTC (SEQ ID NO: 10) |
| | R) | Exon 10 | AGCCTGGGCATCCTTGAGTTCC (SEQ ID NO: 11) |
| β (beta) | (F) | Exon 9 | Same as p53α (F) |
| | (R) | (Exon 9β) | TCATAGAACCATTTTCATGCTCTCTT (SEQ ID NO: 12) |
| γ (gamma) | (F) | Exon 9 | Same as p53α (F) |
| | (R) | (Exon 9γ) | TCAACTTACGACGAGTTTATCAGGAA (SEQ ID NO: 13) |
| E8/9 | (F) | Exon 8 | GAAGAGAATCTCCGCAAGAAAGG (SEQ ID NO: 14) |
| | (R) | Exon 9 | TCCATCCAGTGGTTTCTTCTTTG (SEQ ID NO: 15) |

### Example 3: Serial Humanization of an Autosomal Gene, TMPRSS2, in ACE2 Humanized mESCs, Demonstrating Rapid Production of Multi-Gene Humanized GEMM (Genetically Engineered Mouse Model)

The SARS-COV-2 infection process is mediated not only by the binding of the SARS-COV-2 spike(S) protein to the host cell receptor angiotensin converting enzyme 2 (ACE2), and importantly, the cleavage of the S protein by proteases such as transmembrane protease serine 2 (TMPRSS2) from the host cells. Numerous studies have shown that co-expression of ACE2 and TMPRSS2 in lung epithelial cells is a prerequisite for effective infection. We therefore hypothesized that fully humanized TMPRSS2 on top of hACE2 will better recapitulate physiological human-specific expression patterns in mice, thus improving the accuracy of COVID-19 modeling. Also, humanizing TMPRSS2 may facilitate the development of therapeutics that block the activity of TMPRSS2 in human. This is the first demonstration of serial humanization using mSwAP-In, allowing us to rapidly and directly generate ACE2+TMPRSS2 humanized genetically engineered mouse models (GEMMs) without any mouse crossing when combined with tetraploid complementation approach.

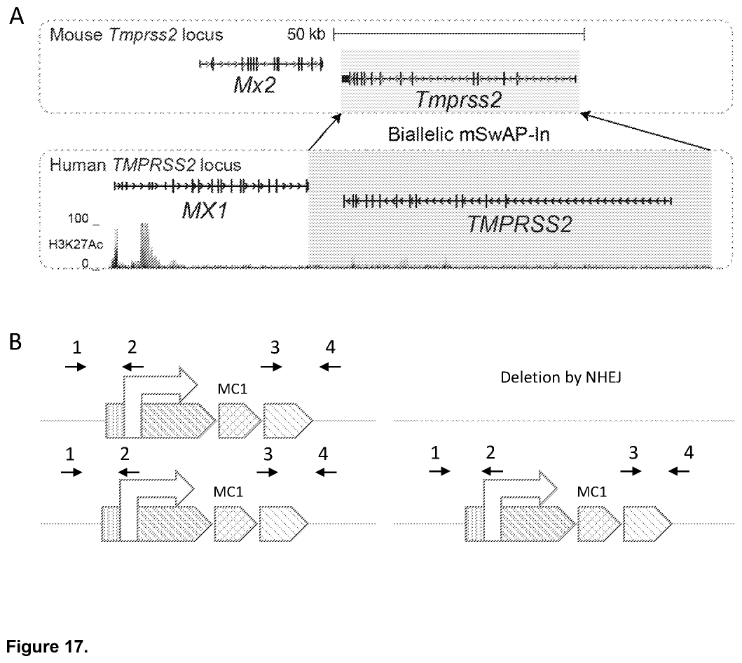

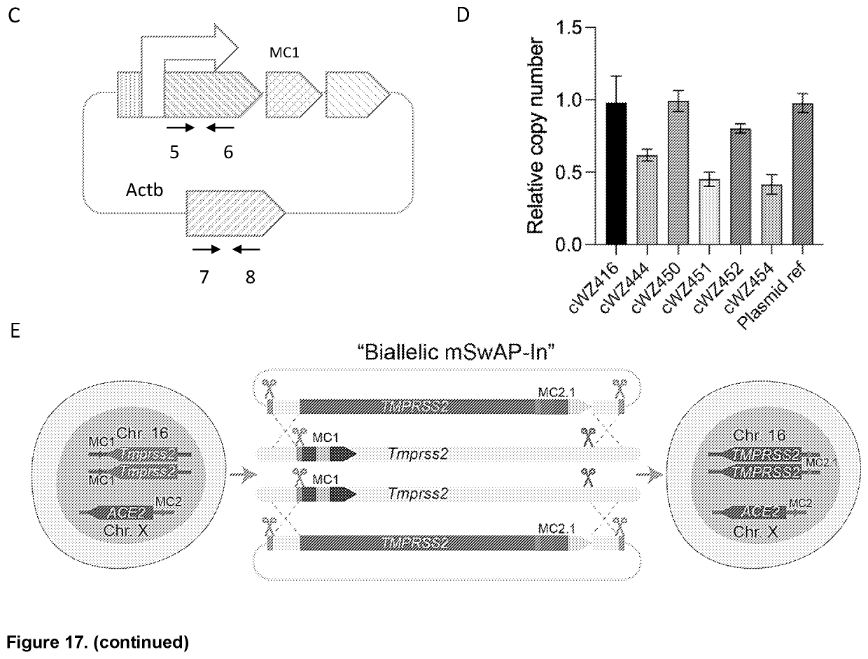

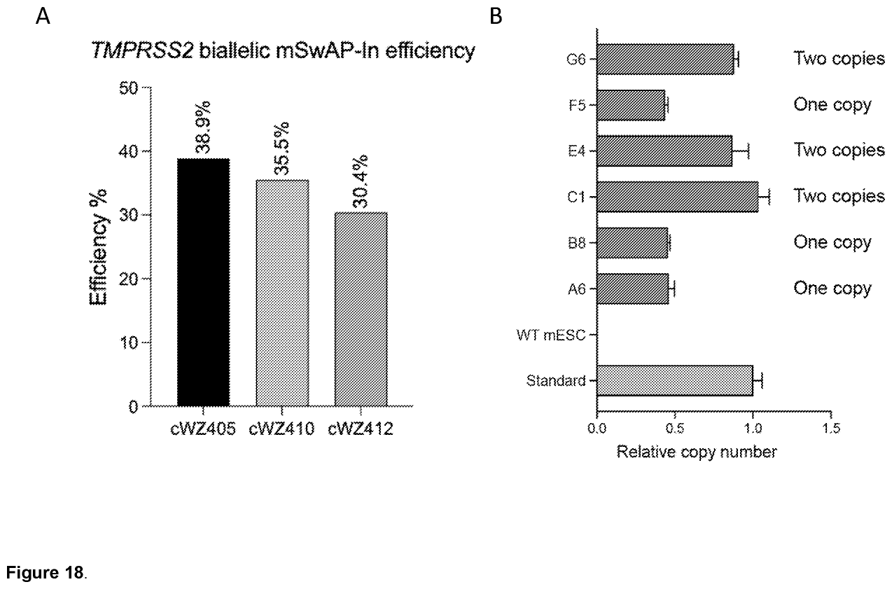

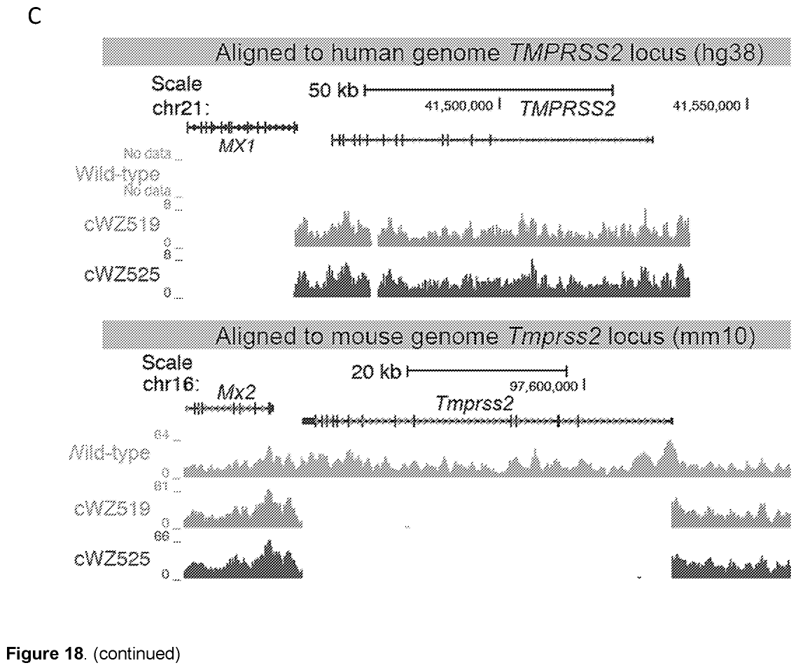

We used the 3′ end of the MXI gene as the left boundary, which is about 5 kb away from the 3′UTR of TMPRSS2 gene. For the right boundary, we included an additional ˜15 kb of TMPRSS2 upstream genomic sequence that contains a putative TMPRSS2 enhancer. The total length of the payload was ˜74 kb (chr21: 41,458,780-41,532,725, hg38) (FIG. 17 panel A). To build the TMPRSS2 payload for mSwAP-In, we digested the wild-type human TMPRSS2 region of interest from CH17-339H2 BAC, which was subsequently assembled in a mSwAP-In compatible vector in yeast, and the payload DNA was fully verified by next-gen sequencing. Since the MC1 inserted in Ace 2 locus was removed during hACE2 humanization by mSwAP-In, we can thus reuse MC1 to generate founder lines for TMPRSS2 humanization. We inserted MC1 downstream of mouse Tmprss2 gene on both alleles of hACE2 mESCs. 26 of 144 clones had both left (primer 1-2) and right junctions (primer 3-4), and lack the wild-type amplicon (primer 1-4). There are two MC1 insertion scenarios that produce the same aforementioned genotyping PCR result, one is biallelic MC1 insertion, the other one is monoallelic MC1 insertion coupled with a large deletion on other allele by non-homologous end joining DNA repair mechanism (FIG. 17 panel B). To distinguish the two scenarios, we constructed a reference plasmid with one copy of MC1 and one copy of Actb gene (FIG. 17 panel C). By performing MC1 copy number qPCR, 3 of the 6 clones have two copies of MC1 (FIG. 17 panel D). Starting with three biallelic MC1 founder lines, biallelic mSwAP-In was performed to humanize TMPRSS2 gene in 116 kb hACE2 mESCs (FIG. 17 panel E). mES clones were initially verified by genotyping PCR of human TMPRSS2 and mouse Tmprss2 genes, as well as the PCR for detecting hemizygous clones. Above 30% success rates were obtained using all three MC1 founder lines (FIG. 18 panel A). TMPRSS2 humanized mESC candidates were further verified by copy number qPCR (FIG. 18 panel B), and capture-sequencing to ensure accurate integration (FIG. 18 panel C).

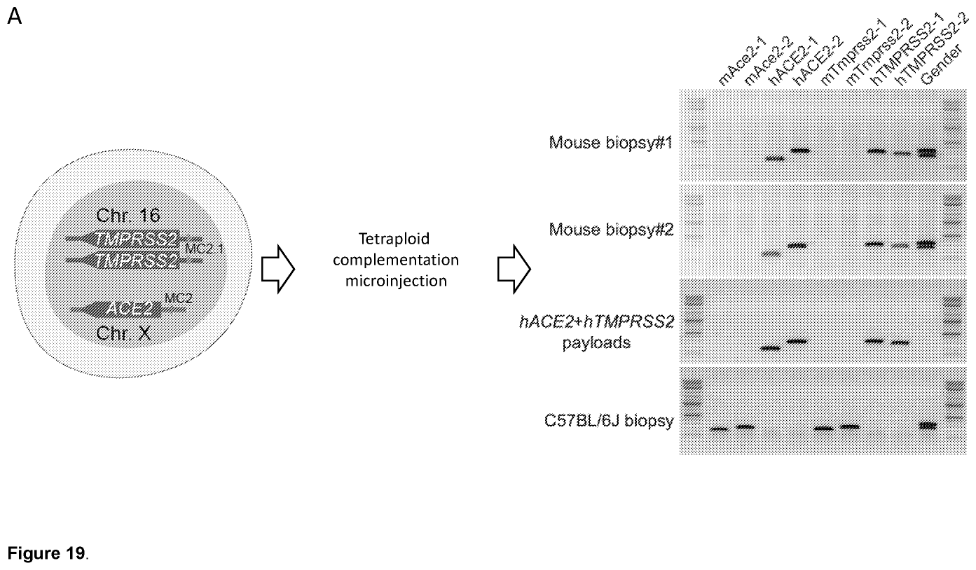

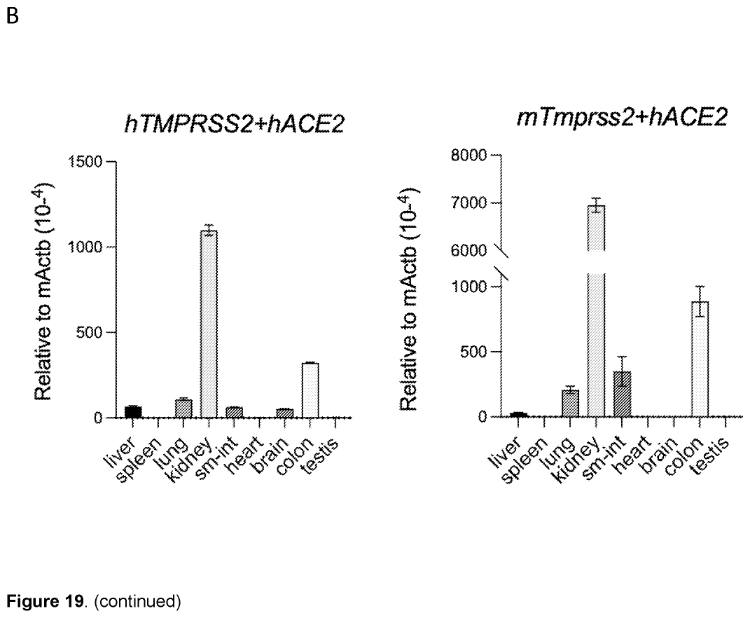

ACE2+TMPRSS2 humanized mouse pups were successfully obtained from tetraploid complementation, demonstrating extensive culturing of mESCs does not significantly impair the ability of mouse development from mESCs. Mouse biopsy genotyping PCR result shows both ACE2 and TMPRSS2 are humanized (FIG. 19 panel A). Moreover, human TMPRSS2 expression was detected by RT-qPCR in liver, lung, kidney, small intestine, brain and colon (FIG. 19 panel B, left), a similar expression pattern as mouse Tmprss2 in hACE2 only mouse, also with the exception of brain expression only seen in TMPRSS2 humanized mouse (FIG. 19 panel B, right).

### Example 4: The Native Mouse Trp53 Tumor Suppressor Gene is Replaced with the “CG-Less” Synthetic Copies of Trp53. Demonstration of a Second Round of mSwAP-In by Overwriting Trp53 Downstream Genes in Various Length

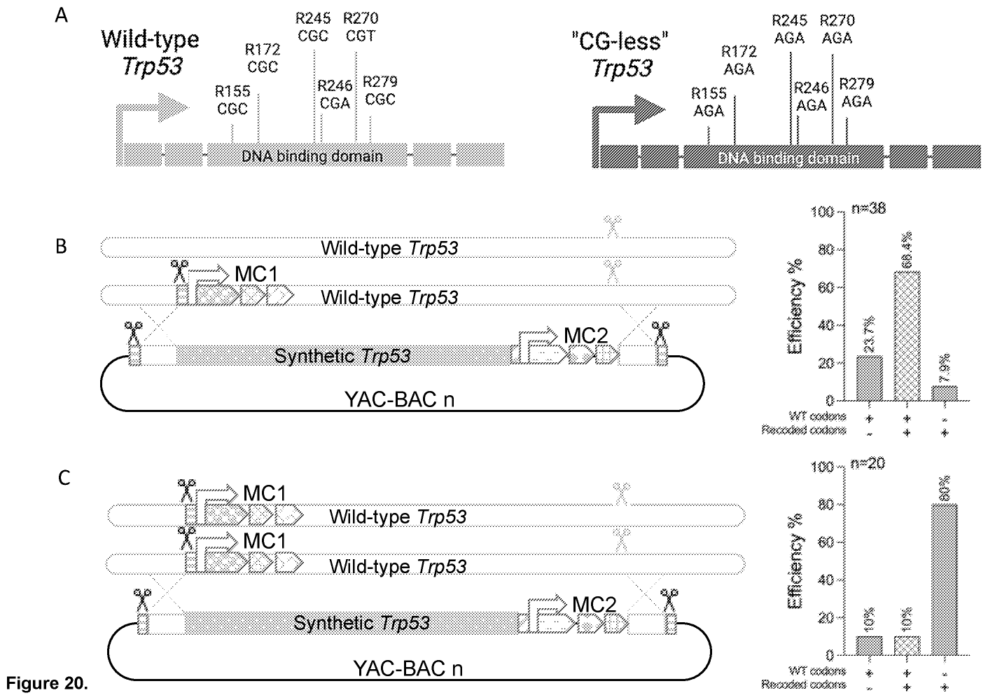

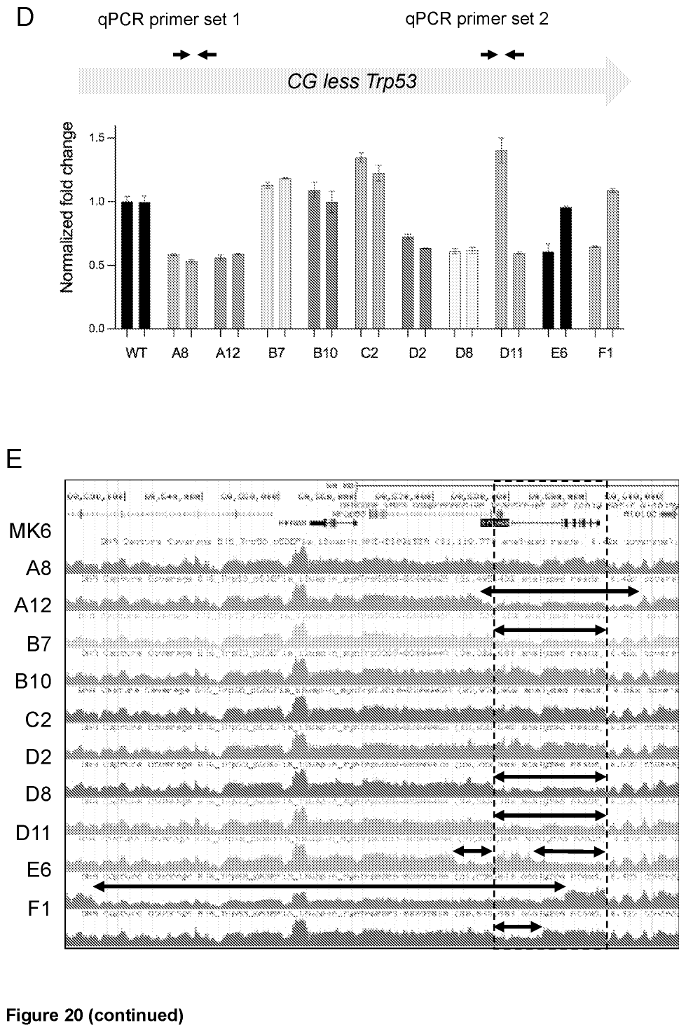

We constructed a synthetic version of Trp53 (synTrp53) that recodes six CG dinucleotides to AG in p53 mutation hotspots (R155, R172, R245, R246, R270, R279) to prevent deleterious (i.e. cancer) mutations (FIG. 20 panel A). When we inserted marker cassette 1 in one of the alleles upstream of Trp53, after deploying mSwAP-In, we found that 87.1% of the colonies lost MC1 and gained MC2 by performing genotyping PCR (n=132). We Sanger sequenced 38 genotype-verified clones and discovered that 26 of them carried the recoded codons in one of the two alleles, 9 of them were unedited, and surprisingly, 3 of them only carried recoded codons (FIG. 20 panel B). When we inserted MC1 in biallelically upstream of Trp53, through mSwAP-In, wild-type Trp53 was overwritten by synTrp53 heterozygously at a rate of 10% (n=20) and homozygously at a rate of 80% (n=20) (FIG. 20 panel C). To determine the copy number of the synTrp53 delivered by mSwAP-In, we performed genomic DNA qPCR assay using two sets of primers, each towards one end of the synTrp53 gene; candidate clones showing near equal copy number as wild-type control were considered biallelic mSwAP-In candidates (FIG. 20 panel D). We also performed capture sequencing for the candidates we identified in FIG. 20 panel D; the capture sequencing result reveals outcomes consistent with the qPCR assay, in which the normalized fold change (either 0.5× or 1× of the control) corresponds to the coverage of the capture sequencing (FIG. 20 panel E).

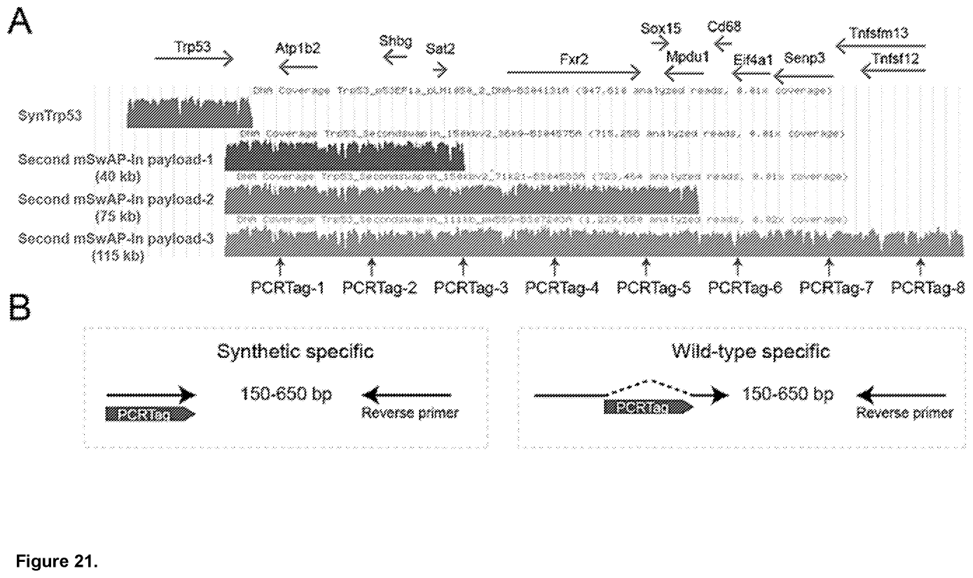

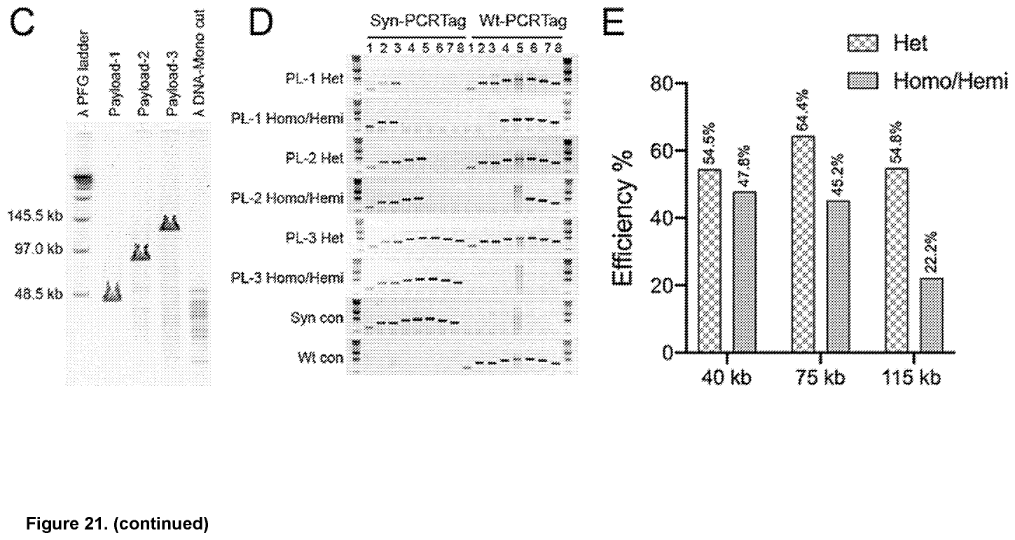

In order to test whether mSwAP-In is iterable, and also probe a minimum upper length limit for a single step of mSwAP-In, we designed 40 kb, 75 kb and 115 kb payloads (PLs) downstream of Trp53 gene for the second round of mSwAP-In (FIG. 21 panel A). To distinguish the synthetic DNA from the wild type, and to further facilitate clone identification, we inserted readily detectable “watermarks” that are evenly distributed across the constructs (every ˜13 kb interspersed the constructs). The watermarks are referred to as mPCRTags because they are similar in principle to previously used PCRTags deployed in the synthetic yeast (Sc2.0) genome, but different in that they were systematically inserted into introns and intergenic regions. The mPCRTags are 28-bp nucleotide sequences that lack homology to the mouse genome. Taking advantage of these PCRTags, we designed synthetic- or wild-type-specific primer pairs (FIG. 21 panel B). Three payload DNAs were assembled by using yeast homologous recombination and verified by sequencing and restriction enzyme digestion (FIG. 21 panel C). We tested both heterozygous and homozygous/hemizygous mSwAP-In using these three payloads, the mPCRTags incorporation indicate the wild-type genome was overwritten by the synthetic payloads either homozygously or hemizygously. The patterns of mPCRTag amplification for heterozygous and homozygous/hemizygous mSwAP-In clones are shown in FIG. 21 panel D. Overall, for heterozygous mSwAP-In, we observed a >50% efficiency for all three payload integrations (n=46). The efficiency did not drop substantially as the payload size increase. Remarkably, for homozygous/hemizygous mSwAP-In, the efficiency for the 40 kb, 75 kb and 115 kb payloads were 47.8%, 45.2% and 22.2% respectively (n=46 for each group) (FIG. 21 panel E).

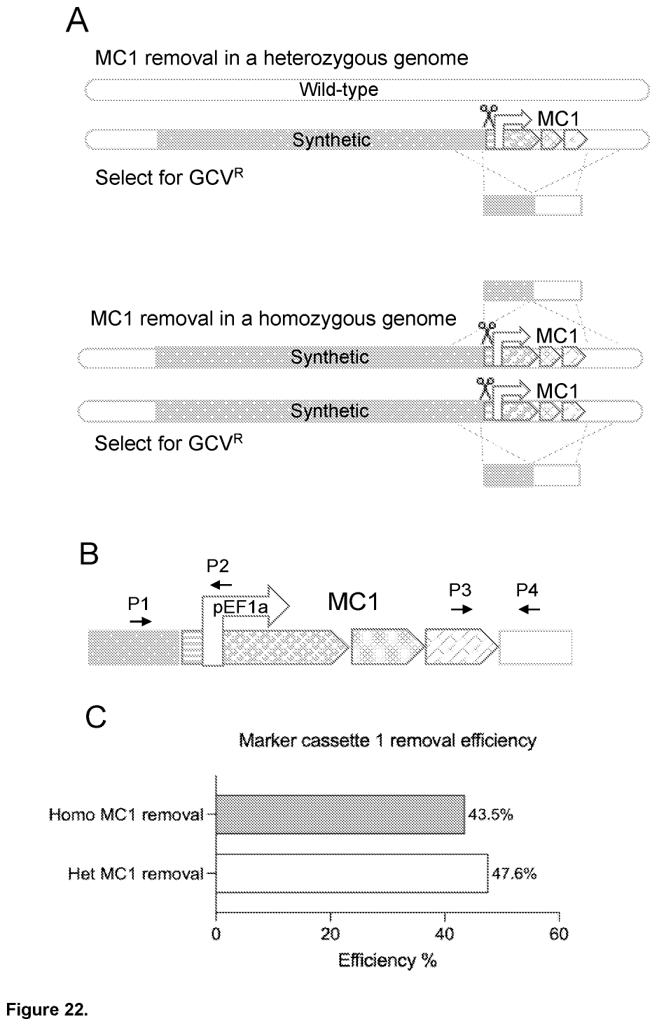

The final (but optional) step of mSwAP-In comprises removing the last used marker cassette from the genome after all the mSwAP-In rewriting steps are finished. Here, we used two strains from the second round mSwAP-In clones shown in FIG. 21 to demonstrate the feasibility of MC1 removal in both heterozygous and homozygous mSwAP-In edited genomes. We deployed a zinc finger nuclease to cut the UGT1 site and a Cas9-gRNA to cut the SV40 terminator in MC1 in both strains, while providing a ˜2 kb repair template as a circular plasmid into the mESCs (FIG. 22 panel A). By applying ganciclovir negative selection, followed by genotyping PCR, we observed 47.6% (n=21) success rate for heterozygous MC1 removal, 43.5% (n=23) for homozygous MC1 removal (FIG. 22 panels B-C).

TABLE 3
guide RNA sequences used in the Examples.

| Guide RNA purpose | Sequence |
|---|---|
| gRNA targeting UGT1 | GCUUCAUGUGGUCGGGGUAG (SEQ ID NO: 16) |
| gRNA targeting UGT2 | CACGAGGGUGGGCCAGGGCA (SEQ ID NO: 17) |
| gRNA mediates MC1 insertion for ACE2 | AGGGUCUUCUCUACUCAAGG (SEQ ID NO: 18) |
| humanization | |
| Custom gRNA for ACE2 humanization | UUAUUACUAGAGUAGCAGGG (SEQ ID NO: 19) |
| gRNA mediating MC1 insertion for | GGAGCAGAUGGUCACUCCAG (SEQ ID NO: 20) |
| TP53-WRAP53 humanization | |
| Custom gRNA for TP53-WRAP53 | GUCAUUGUGUCUGUUCGGGG (SEQ ID NO: 21) |
| humanization | |
| gRNA mediating MC1 insertion for | UACUGCCGUGUAUCGUAUUG (SEQ ID NO: 22) |
| “CG-less” Trp53 engineering | |
| Custom gRNA for synTrp53 engineering | UUGUAUAGGACCCUCGGGCA (SEQ ID NO: 23) |
| Custom gRNA for engineering the 40 kb | CAUCUCACCAGCCUAGCAGG (SEQ ID NO: 24) |
| payload downstream of synTrp53 | |
| Custom gRNA for engineering the 75 kb | UCAUUAACCCAGGAGCCACG (SEQ ID NO: 25) |
| payload downstream of synTrp53 | |
| Custom gRNA for engineering the 115 | ACCUGCUUCACAGAUAACUG (SEQ ID NO: 26) |
| kb payload downstream of synTrp53 | |

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. A composition comprising a first contiguous DNA molecule comprising:
(i) a first homology segment that is homologous to a first segment of a chromosome;
(ii) a first marker cassette (MC1) comprising a first guide RNA (gRNA) target site (UGT1);
(iii) a second homology segment that is homologous to a second segment of the chromosome;
wherein the first gRNA target site is not endogenous to the genome of a cell prior to introducing the MC1 to the cell.

2. A composition comprising a first contiguous DNA molecule comprising:
(i) a first homology segment that is homologous to a first segment of a chromosome;
(ii) a first marker cassette (MC1) comprising a first guide RNA (gRNA) target site (UGT1);
(iii) a second homology segment that is homologous to a second segment of the chromosome;
wherein the first gRNA target site is not endogenous to the genome of a cell prior to introducing the MC1 to the cell; and
a second contiguous DNA molecule comprising:
(i) a copy of the first gRNA target site;
(ii) the first homology segment;
(iii) a first heterologous DNA payload; and
a second marker cassette (MC2) comprising;
(i) a second gRNA target site (UGT2) that is different than the first gRNA target site; and
a third homology segment.

3. The composition of claim 2, wherein the composition further comprises a third contiguous DNA molecule comprising in a 5′-3′ direction:
(i) a copy of the UGT2;
(ii) a fourth homology segment;
(iii) a second heterologous DNA payload;
(iv) a copy of the MC1
(v) a fifth homology segment; and
(vi) a second copy of the UGT2.

4. The composition of claim 2, wherein the composition is introduced into a cell.

5. The composition of claim 4, wherein the cell is a mammalian cell.

6. The composition claim 4, wherein the composition is introduced into an endogenous chromosome segment of the cell.

7. The composition of claim 2, wherein UGT1 and/or UGT2 is not endogenous to the endogenous chromosome segment of the cell prior to introduction of the composition into the cell.

8. The composition of claim 2, wherein the composition further comprises a UGT1 independent nuclease that cleaves a segment of the chromosome that is upstream of and not present in the third homology segment or the DNA payload.

9. The composition of claim 2, wherein the composition further comprises a UGT1 gRNA-directed nuclease.

10. The composition of claim 2, wherein introduction of the UGT1 gRNA-directed nuclease promotes homologous recombination based replacement of the entire MC1 via the first homology segment and the third homology segment.

11. The composition of claim 2, wherein MC1 comprises a promoter, a first reporter protein, a first positive selection marker, and/or a first negative selection marker.

12. The composition of claim 2, wherein MC2 comprises a promoter, a second reporter protein, a second negative selection marker, and/or a second positive selection marker.

13. The composition of claim 2, wherein MC1 comprises a promoter, a first reporter protein, a first positive selection marker, and/or a first negative selection marker, and wherein MC2 comprises a promoter, a second reporter protein, a second negative selection marker, and/or a second positive selection marker.

14. The composition of claim 13, wherein the first reporter protein, the first positive selection marker and/or the first negative selection marker of MC1 is different than the second reporter protein, the second positive selection marker, and/or the second negative selection marker of MC2.

15. The composition of claim 13, wherein the promoter of MC1 and/or MC2 are the same promoter.

16. The composition of claim 13, wherein the first reporter protein, the first positive selection marker and/or the first negative selection marker of MC1 is the same as the second reporter protein, the second positive selection marker, and/or the second negative selection marker of MC2.

17. The composition of claim 13, wherein the first, and/or second reporter protein comprises a fluorescent protein.

18. The composition of claim 13, wherein the first and/or second positive selection marker comprises an antibiotic resistance gene.

19. The composition of claim 18, wherein the antibiotic resistance gene comprises N-acetyltransferase (pac), Blasticidin S deaminase (bsd), Neomycin (G418) resistance gene (neo), Hygromycin resistance gene (hygB), Zeocin resistance gene (Sh ble), or hypoxanthine phosphoribosyltransferase 1 gene (HPRT1).

20. The composition of claim 13, wherein the first and/or second negative selection marker comprises a toxin-antitoxin system, or a counter selection.

21. The composition of claim 13, wherein the first and/or second negative selection marker comprises herpes simplex virus type 1-thymidine kinase (HSV1-TK), human HPRT1, or the cytosine deaminase gene (codA).

22. The composition of claim 2, wherein the first homology segment is upstream of the endogenous gene to be replaced and the second homology segment is downstream of the endogenous gene to be replaced.

23. The composition of claim 2, wherein the first homology segment is downstream of the endogenous gene to be replaced and the second homology segment is upstream of the endogenous gene to be replaced.

24. The composition of claim 2, wherein the cell comprising the first heterologous DNA payload and MC2 is selected using the second positive and first negative selection based on the second positive (MC2) and first (MC1) negative selection markers and optionally one or both reporter proteins.

25. The composition of claim 3, wherein the cell comprising the second heterologous DNA payload and MC1 is selected using the first positive (MC1) and second negative (MC2) selection based on the first positive and second negative selection markers.

26. The composition of claim 2, wherein the heterologous DNA payload is only inserted into one homologous chromosome of the cell to thereby provide a heterozygous or hemizygous chromosome pair in which only one chromosome in the pair comprises the heterologous DNA payload.

27. The composition of claim 2, wherein the heterologous DNA payload is inserted into both homologous chromosomes of the cell to thereby provide a homozygous chromosome pair in which both chromosomes in the pair comprises the heterologous DNA payload.

28. The composition of claim 2, wherein the heterologous DNA payload comprises a gene.

29. The composition of claim 2, wherein the cell comprises a human cell, or a non-human cell.

30. The composition of claim 2, wherein the cell comprises a totipotent stem cell, a pluripotent stem cell, a multipotent stem cell, or an oligopotent stem cell.

31. The composition of claim 29, wherein the non-human cell comprises a mouse stem cell which is optionally a mouse embryonic stem cell.

32. The composition of claim 31, wherein the mouse embryonic stem cell is diploid (2n) and is optionally present in or is introduced into a tetraploid (4n) mouse blastocyst.

33. A pseudopregnant mouse comprising the blastocyst of claim 32.

34. A mouse obtained from the pseudopregnant mouse of claim 33, and/or a progeny of the pseudopregnant mouse.

35. An isolated DNA comprising the recombinant polynucleic acids encoding MC1 and/or MC2 of claim 2.

36. An expression vector encoding MC1 and/or MC2 of claim 2.

37. A kit comprising a DNA molecule comprising an MC1 or an MC2 of claim 2, or a combination of DNA molecules comprising the MC1 and the MC2.

38. The kit of claim 37, further comprising a guide RNA or a vector encoding the guide RNA, said guide RNA being targeted to a UGT1 or UGT2, or a combination of said guide RNAs, or one or more vectors encoding the combination of guide RNAs.

39. The kit of claim 37, further comprising a nuclease, or a vector encoding the nuclease, wherein the nuclease is specific for the MC1 or the MC2, or a combination of said nucleases.

40. The kit of claim 39, further comprising at least one agent that can be used for selection based on the positive or negative selection markers, or a combination of said agents.

41. A mouse obtained from the pseudopregnant mouse of claim 33 and/or a progeny of the pseudopregnant mouse, wherein the first or the second payload encodes an angiotensin converting enzyme 2 (ACE2).

42. The mouse of claim 41, wherein the ACE2 is a human ACE2.

43. The heterologous DNA payload of claim 2, wherein the payload is a human angiotensin converting enzyme 2 (ACE2).

44. A pharmaceutical composition comprising the composition of claim 2, and a pharmaceutically acceptable excipient or carrier.

45. A method of treating a disease in a subject in need thereof, comprising administering a therapeutically effective amount of the pharmaceutical composition of claim 44.

46. A method of modifying a chromosome in a mammalian cell to replace an endogenous chromosome segment with a heterologous chromosome segment, comprising administering the composition of claim 2 to the cell.

47. The method of claim 46, further comprising iteratively administering additional heterologous DNA payloads to the cell to replace the endogenous chromosome segment, wherein the additional heterologous DNA payloads comprise MC1 if MC2 was administered previously, and/or MC2 if MC1 was administered previously.

48. A mouse obtained from the pseudopregnant mouse of claim 33 and/or a progeny of the pseudopregnant mouse, wherein the first or the second payload encodes an angiotensin converting enzyme 2 (ACE2) and wherein the first or second payload also comprises a human TMPRSS2 gene.

49. A mouse obtained from the pseudopregnant mouse of claim 33 and/or progeny of said mouse, wherein the first or second payload comprises human TP53 and WRAP53 genes.
